(19) United States
(12) Patent Application Publication
(10) Pub. No.: US 2015/0064780 A1
(43) Pub. Date: Mar. 5, 2015

(54) DISPOSABLE SINGLE USE SELF-CONTAINED CYCLIC PRESSURE AND FLOW BIOREACTOR SYSTEM

(71) Applicants: Richard Hopkins, Kansas City, MO (US); Gabriel Converse, Kansas City, MO (US); Eric Buse, Kansas City, MO (US)

(72) Inventors: Richard Hopkins, Kansas City, MO (US); Gabriel Converse, Kansas City, MO (US); Eric Buse, Kansas City, MO (US)

(73) Assignee: THE CHILDREN'S MERCY HOSPITAL, Kansas City, MO (US)

(21) Appl. No.: 14/390,170

(22) PCT Filed: Apr. 2, 2013
(86) PCT No.: PCT/US13/35018

§ 371 Date: Oct. 2, 2014

Related U.S. Application Data
(60) Provisional application No. 61/619,287, filed on Apr. 2, 2012.
(60) Provisional application No. 61/765,994, filed on Feb. 18, 2013.

(51) Int. Cl. C12N 5/071

(52) U.S. Cl. CPC C12N 5/069
USPC 435/325; 435/294.1

(57) ABSTRACT

The present invention provides for a bioreactor system (3, 4) that is suited for one time use. The bioreactor system (3, 4) comprises a first bioreactor vessel (1), a second bioreactor vessel (20), a tissue retainer (35), and a cap (40). Advantageously, the bioreactor system (3, 4) of the present invention provides a sterile environment that is maintained over the course of cell seeding applications.

### RELATED APPLICATIONS

This application claims priority to U.S. Provisional Patent Application Nos. 61/619,287, filed on Apr. 2, 2012 and Application No. 61/765,994, filed on Feb. 18, 2013, the teaching and contents of which are hereby incorporated by reference.

### BACKGROUND OF INVENTION

Numerous types of tissue engineered constructs and vascular grafts have been produced over the last few decades. Previous tissue constructs have included man-made polymers as substitutes for various portions of the organ to which the tissue belongs. Materials such as Teflon and Dacron have been used in various configurations including scaffoldings, tissue engineered blood vessels, and the like. Nanofiber self-assemblies have been used as microscaffolds upon which cells are grown. Textile technologies have been used in the preparation of non-woven meshes made of different polymers. The drawback to these types of technologies is that it is difficult to obtain high porosity and a regular pore size, which contributes to unsuccessful cell seeding. Currently approved clinical biological/bioprosthetic heart valve replacement options (allografts and xenografts) often result in reduced durability (likely due to innate inflammation and immune rejection and consequential calcification), ultimately leading to accelerated failure.

Heart valve disorders, whether congenital or degenerative, are common both in the United States and worldwide. A variety of options are available for valve replacement, including mechanical valves, bioprosthetic xenografts and cryopreserved homografts; however these all suffer from deficiencies. Mechanical valve substitutes require lifelong anticoagulation therapy, while bioprosthetic xenografts only offer a 10-15 year service life in the adult population due to calcification or structural fatigue. Calcification of bioprosthetic valve substitutes is accelerated in the pediatric population, further reducing the expected life of the valve before replacement. The cryopreserved homograft is used most frequently for heart valve replacement in the pediatric population. While offering excellent hemodynamic performance, small diameter homografts are limited in availability and are susceptible to fibrosis and calcification. As with mechanical and bioprosthetic valves, the cryopreserved homograft does not offer somatic growth, meaning multiple revision surgeries are required throughout the patient's lifetime.

The tissue engineered heart valve (TEHV) overcomes the problems inherent in the prior art and provides a distinct advance in the state of the art. This is especially advantageous in the pediatric population, in which initial intervention using a living, growing valve would eliminate the need for multiple revision surgeries as the child matures. However, in addition to the science associated with heart valve tissue engineering, logistical and regulatory challenges must be overcome if clinically useful solutions are to be realized.

Despite advances in the laboratory, a clinically useful, living heart valve is still needed in the art. Many of the factors that have limited translation from laboratory success to clinical success also prevent processing of the TEHV in the clinical setting including 1) the use of complex bioreactor systems that are not single-use or patient-specific, 2) the absence of an ideal clinically available, patient-specific cell source and 3) the use of extended ex vivo seeding protocols. Selection of the ideal scaffold for cell seeding is also difficult and has hampered development, though this does not directly relate to processing in the clinical environment.

Bioreactors have been developed for the use of heart valve tissue engineering as well as for other tissue engineering applications. Various strategies have been employed to create a personalized bioreactor and the majority of systems described previously have attempted to mimic in vivo conditions. This typically involves fluid flow through peripheral chambers intended to mimic the function/effects of the various heart chambers and systemic pulmonary circulations via bulky and technically awkward flow loops. However, these types of systems have been unsuccessful in providing tissue engineered constructs that thrive once implanted into the intended recipient. Additionally, systems described in the literature are not intended for single use and are not intended to be disposable, and thus intended for repeated seeding/sterilization cycles. The bioreactors previously described in the art have several drawbacks including the use of a bioreactor more than one time and patient specific bioreactors. In this regard, FDA has expressed concerns over the repeated, clinical use of bioreactors, especially for use in connection with tissue engineered heart valves.

Numerous bioreactor systems have been reported in the literature. In vitro re-endothelization of decellularized valve constructs is often demonstrated; however, infiltration of seeded cells into the cuspal tissue solely through the use of bioreactor based seeding and conditioning has proven difficult. This is a monumental challenge facing the use of decellularized heart valve as a scaffold for tissue engineering applications. Additionally, while the FDA has not yet issued a guidance document concerning the regulation of the TEHV or associated bioreactor systems, mitigating the potential for disease transmission during processing will certainly aid in gaining regulatory acceptance. Previous bioreactor systems are not single-use, patient-specific systems, raising serious concerns regarding sterility of the bioreactor and the potential for disease transmission during the seeding and conditioning process.

Accordingly, what is needed in the prior art is a bioreactor system that can be used to efficiently recellularize a decellularized tissue. Further, what is needed is a bioreactor that is appropriate for use in a clinical setting and is disposable in order to avoid the problematic issues identified by the FDA that possibly occur when a bioreactor is used multiple times. In addition, a bioreactor is needed that can recellularize a tissue with a greater population of cells below the basement membrane to provide for a great opportunity for recellularization of the tissue when it is implanted into the recipient. Further, the prospect of tissue engineering within the hospital environment offers clear advantages, as this would simplify the collection of recipient-specific cells and would allow clinicians to maintain better control over harvested cells because transfer to an off-site facility for processing would not be required.

### SUMMARY OF THE INVENTION

The present invention overcomes the obstacles of the prior art and provides for a bioreactor that is specifically designed for clinical use and provides an optimal environment for cell seeding and tissue conditioning for tissue engineering applications. Advantageously, the bioreactor of the present invention provides a single use self-contained system allowing the environment to maintain sterility and avoiding some of the concerns the FDA has addressed in previous systems. The present invention also provides for a bioreactor cap capable of maintaining sterility when a tissue is transferred from one bioreactor chamber to another. Finally, a bioreactor vessel is provided that promotes better cell seeding of the tissue in the bioreactor.

Cyclic pressure and flow waveforms are imposed in the system of the present invention on fluid (specialized media optimal for the specific cells and scaffold tissues) with a gas interface (composition optimized for specific cells and scaffold tissues), and which is thus transmitted to the constructs to be tissue engineered. This system is fully adjustable by the operator in overall magnitude and time (rate and length of cycles) and can be adjusted within the bioreactor to affect the entire construct, or subregions of the constructs to be tissue engineered. This biological and mechanical conditioning is accomplished by creating hydraulic loading with or without regional discontinuities across tissue planes, or inside or outside tubular or cavitary structures that are extensively tunable or adjustable such that negative and positive gradients can be created as desired and with whatever time dependent parameters desired.

The bioreactor system of the present invention preferably comprises a first bioreactor vessel, a second bioreactor vessel, a cap, and a grip.

The bioreactor vessels are preferably sized appropriately for a tissue to be housed within the bioreactor system. The bioreactor system preferably comprises a first bioreactor vessel having a cylindrical chamber, with two opposed ends and a continuous cylindrical side wall. Preferably, the first bioreactor vessel has an opening at the proximal end. The distal end of the first bioreactor vessel is preferably closed, where the closed end preferably gradually narrows in diameter on an angle away from the cylindrical sidewall to a generally flat plane having a surface area less than that of the proximal opening at the other end of the first bioreactor vessel. It is preferred that the closed end of the first bioreactor vessel provides a narrowed bottom portion allowing any fluid material within the retainer body to collect or concentrate at the bottom or at the distal end. Advantageously, this allows for the use of fewer cells, (which during clinical applications may be quantitatively limited and thus quite precious) and less media when using the bioreactor system for seeding cells onto a biological construct and provides a better environment for cell seeding of a tissue construct. In a preferred embodiment, the bioreactor vessel has one or more ports on the continuous cylindrical side wall, wherein the ports provide a passageway from the interior of the vessel to the exterior of the vessel. In preferred forms, the first bioreactor vessel has a beveled edge along the opening on the proximal end such that the beveled edge engages a cap, attachably sealing the cap and bioreactor vessel. In a more preferred embodiment, the bioreactor vessel comprises a stepped portion on the proximal end, such that there are two beveled edges that have the ability to engage the bioreactor cap forming a seal separating a sterile interior “zone” from a clean exterior “zone”.

The distal end of the first bioreactor vessel preferably has a diameter that is smaller than that of the proximal end. The diameter of the distal end of the first bioreactor vessel is preferably between 1% to 80% smaller in diameter than that of the proximal end, more preferably, the distal end is between 5% and 70% smaller, still more preferably, the distal end is between 15% and 60% smaller, still more preferably, the distal end is between 20% to 55% smaller, even more preferably between 35-53% smaller, and most preferably, the distal end is about 51% smaller. As noted above, the vessels can be sized to accommodate any tissue therein. In some preferred forms, the diameter of the distal end of the first bioreactor vessel is preferably between about 0.5 and about 36 inches, where the diameter of the proximal end of the bioreactor vessel is between 0.75 and about 48 inches. The diameter of the distal end can be adjusted depending on the type and size of the tissue used for the bioreactor system. For heart valve applications, preferred diameter sizes of the distal end range from about 0.5 to about 5 inches, more preferably between about 1 to 4 inches, and still more preferably between about 1.25 to about 3 inches, even more preferably between about 1.4 to about 2 inches, and most preferably about 1.6 inches. Similarly, preferred diameter sizes of the proximal end of the first bioreactor vessel are between about 0.75 inches to about 7.5 inches, more preferably between about 1.5 to 6 inches, and still more preferably between about 2.5 to about 5 inches, even more preferably between about 2.8 to about 4.2 inches, and most preferably around 3.125 inches. The diameter of the distal end of the first bioreactor vessel preferably narrows or decreases at an angle from the continuous cylindrical side wall of the first bioreactor vessel. This angle is preferably from about 15° to about 70°, more preferably from about 25° to about 60°, still more preferably from about 35° to about 50°, and most preferably, at about a 45° angle relative to the side wall of the bioreactor vessel. In a most preferred embodiment, where the distal end of the first bioreactor vessel has a diameter of 1.6 inches, and the proximal end has a diameter of 3.125 inches, the diameter of the distal end increases to 2.491 inches over a vertical height increase of 0.535 inches. Thus, ratios in these dimensions are within the scope of this invention. In an alternate embodiment, a standard female luer connection is molded into the angled distal end of the first bioreactor vessel to aid in focused cell seeding. Advantageously, in this embodiment, where a heart valve is used, this embodiment permits the addition of bone marrow derived cells, or other sourced cells (e.g. umbilical cord, stem cells, allogeneic cells, genomically manipulated cells, and the like) in close proximity to the valve annulus.

The first bioreactor vessel may be made of any material suitable for use with biologic applications. Preferably, the first bioreactor vessel is made from an injection molded polymer. It is preferred that the polymer is a low attachment polymer, even more preferably, the low attachment polymer is bacteriological grade polystyrene. The use of a low attachment polymer is to reduce the potential for cell attachment to the internal chamber wall. Additionally, it is preferred that the first bioreactor chamber is molded with a high surface finish.

In a preferred embodiment, the bioreactor system of the present invention additionally comprises a second bioreactor vessel preferably having a cylindrical chamber, with two opposed ends and a continuous cylindrical side wall. In preferred forms, there is an opening on the proximal end that extends between the cylindrical side walls. Preferably, the second bioreactor vessel also has one or more openings on the distal end. The one or more openings on the distal end of the second bioreactor vessel preferably provide access for liquid or gas to enter and exit the bioreactor system. In a preferred embodiment, the distal end of the second bioreactor vessel provides a plurality of openings, through which fluid or gas passes into the bioreactor vessel and out of the bioreactor vessel. Any system known in the art that is capable of moving fluid into a bioreactor vessel and out of a bioreactor vessel can be coupled to the bioreactor system for purposes of the present invention. The use of a system where pressure forces liquid up into the bioreactor vessel and then draws liquid out of the bioreactor vessel is especially preferred. In a preferred embodiment, the bioreactor vessel has one or more ports on the continuous cylindrical side wall. Preferably, the second bioreactor vessel comprises a homing device in the base or distal end of the bioreactor vessel. This homing device preferably attracts a counterpart in the base of a tissue retainer or grip, such that the grip is positioned in a desired location, preferably at the center of the surface area of the base or distal end of the bioreactor vessel when the grip is placed in contact with the distal end of the bioreactor vessel. This homing device is preferably a steel alloy or other type of material that attracts magnets and an attractive magnet, but any homing device could be used for purposes of the present invention. The steel alloy or other type of material is placed in the bottom tissue retainer and the magnet is secured in a holder and affixed to the distal portion of the second bioreactor vessel. Preferably, the second bioreactor vessel has a beveled edge along the opening on the proximal end such that the beveled edge engages a cap, attachably sealing the cap and bioreactor vessel. In a more preferred embodiment, the bioreactor vessel comprises a stepped portion on the proximal end, such that there are two beveled edges that engage the bioreactor cap forming a seal.

As with the first bioreactor, the second bioreactor vessel may be made of any material suitable for use with biologic applications. Preferably, the second bioreactor vessel is made from an injection molded polymer. It is preferred that the polymer is a low attachment polymer, even more preferably, the low attachment polymer is bacteriological grade polystyrene. The use of a low attachment polymer is to reduce the potential for cell attachment to the internal chamber wall. Additionally, it is preferred that the second bioreactor vessel is molded with a high surface finish.

In an alternate embodiment, the second bioreactor vessel is used in combination with bellows. The bellows system preferably drives conditioning media into the second bioreactor. A variety of materials could be used for the bellows system, but in preferred forms, the bellows system includes a blow molded polymer component used to drive conditioning media into the second bioreactor vessel for pulsatile conditioning of a tissue. This embodiment of the bioreactor system is specifically designed without the use of exterior flow loops to provide a pulsatile pressure waveform to the seeded scaffold, providing fluid flow both through and outside the valve scaffold, to secure the proximal end of the scaffold in a stationary position, and to maintain sterility. It is generally positioned at the distal (closest to the bellows) end of the second bioreactor vessel where it is removably attached using any conventional manner. In some preferred forms, the bellows system is threadably engaged with the distal end of the second bioreactor vessel.

The bioreactor system of the present invention preferably comprises a cap that detachably connects to each of the bioreactor vessels. The cap has a surface area that is large enough to cover the proximal opening of each bioreactor vessel for which the cap is being used in connection with. Preferably, the cap has one or more fasteners, clamps, or other attachment mechanisms allowing the cap to detachably connect to the bioreactor vessel. The cap preferably includes two opposed faces with the top face being the farthest from the proximal opening of the bioreactor vessels when the vessel and cap are connected and the bottom face being the closest to the proximal opening of the bioreactor vessels when the vessel and the cap are connected. In some forms, the top face of the cap may comprise one or more holes or ports that extend through the cap to the bottom face. Preferably, these one or more holes or ports allow for the escape of air or gas within the bioreactor vessel, or allow a user to alter the internal pressure or gaseous environment of the bioreactor vessel by altering the number of open/closed ports or by variably constricting effective flow diameters of the ports in any combination to tune outflow resistances and thus shape the magnitude and time morphology of the resultant pressure waveforms experienced by the tissues. In a most preferred embodiment, the cap has a stepped portion with a smaller diameter than that of the overall diameter of the cap. The stepped portion preferably comprises a plurality of holes or ports, which extend between the two faces of the cap and which can be used for the escape of air or gas within the bioreactor vessel or to allow the user to alter the pressure or gaseous environment of the bioreactor vessel. A plug or valve may be used to selectively block the entrance or exit of gas or other materials from each hole or port in the cap. Preferably, a cylindrical side wall extends from the bottom face of the cap having an edge. It is preferred that the edge of the cylindrical side wall comprises a ring of material that engages the bioreactor vessel in such a way to provide a seal when the cap is affixed to the bioreactor vessel. In an embodiment, where the bottom face of the cap comprises a stepped portion, a second ring of material is attached to the stepped portion so that the material engages the bioreactor vessel in such a way as to provide an even more secure seal when the cap is affixed to the bioreactor vessel. The material preferably engages the beveled portions of the bioreactor vessel to form a seal. This embodiment, comprising a double seal between the bioreactor chamber and the cap ensures a sterile environment within the bioreactor vessel and illustrates an advantage over bioreactors in the prior art. The material used to create a seal between the bioreactor cap and vessel is preferably a rubber O-ring, however, any material capable of creating and maintaining a seal can be used for the present invention.

Preferably, the cap additionally comprises an internal elevator mechanism that is attached perpendicularly to the top surface area of the cap. The elevator mechanism can be any known in the art, but is preferably a screw mechanism with a housing. The elevator mechanism is preferably affixed to the cap through a hole in the top face of the cap, where a portion of the elevator mechanism extends through the top face of the cap and into the bioreactor vessel when the cap is affixed to the open end of the bioreactor vessel, such that part of the elevator mechanism is outside of the bioreactor vessel and part of the elevator mechanism is inside the bioreactor vessel. The elevator mechanism preferably threadably engages a guide element directing and allowing the upward or downward motion of the elevator mechanism. This guide element for directing and allowing motion of the elevator mechanism is preferably attached to the bottom face of the cap, such that a user can control and guide the upward or downward motion of the elevator mechanism while the cap is attached to the bioreactor vessel. When the elevator mechanism comprises a screw mechanism, the edges of the elevator mechanism preferably engage a groove on each side of the bioreactor vessel to allow and guide the upward and downward movement of the elevator mechanism in a straight line by inhibiting rotation of the any part of the mechanism other than the screw. The element allowing for a user to control the upward or downward movement of the elevator mechanism can be any conventional apparatus, but is preferably a rotatable knob. The upward and downward controlling element engages the screw mechanism and is preferably located adjacent to the top face of the cap such that a user has access to it and can rotate it to thereby raise and lower the screw without removing the cap. In a preferred embodiment, the elevator mechanism may further comprise a housing that surrounds the body of the elevator mechanism. Preferably, the housing surrounds a screw and turning of the screw allows for the upward and downward motion of the elevator mechanism with repeated adjustments at any time without the outside portion of the elevator mechanism entering the chamber and thus contaminating the interior of the bioreactor. The housing for the elevator assembly is preferably fixably attached to the bottom face of the cap. In a preferred embodiment, the bioreactor cap is transferable between the first and second bioreactor vessels. This advantageously allows a tissue to be transferred from the first bioreactor vessel to the second bioreactor vessel without detaching from the cap and tissue specific adapter grips.

In a preferred embodiment, the elevator mechanism is attached to the bioreactor cap using a series of compression rings and gaskets. Preferably, the elevator mechanism comprises a machined drive screw that extends both above (outside bioreactor vessel) and below (inside bioreactor vessel) the cap. Preferably, a shelf adjustment knob is attached to the section of the drive screw above (outside bioreactor vessel) the top face of the cap. The polymeric elevator is then threaded onto the section of the drive screw below the bottom face of the cap. Upon assembly, the rotational position of the polymeric elevator is fixed through the use of arms that extend out from the centerline and mate with groves in the chamber walls. Thus, as the external adjustment knob is turned, the vertical position of the elevator changes. This allows for placing a tissue in the bioreactor vessel in the center of the vessel or for adjusting the tension on the tissue within the bioreactor system.

In a preferred embodiment of the present invention, the elevator mechanism is coupled to a tissue retainer such that by engaging the elevator mechanism to move in either an upward or downward direction, the tissue retainer moves along with the elevator mechanism. Preferably, a tissue is attached to the tissue retainer such that the elevator mechanism allows movement of the tissue within the bioreactor vessel in an upward or downward motion without altering the sterile interfaces. Preferably, the elevator mechanism contains a through hole at the site of the tissue retainer attachment to allow for access and/or insertion of cells, media, or other therapeutic components. Additionally, the hole provides a point where fluid could pass through and spill over into the rest of the chamber if so desired.

The bioreactor cap can be made of any material suitable for handling biological components, but is preferably an injection molded polymer (e.g. polypropylene). Advantageously, the cap facilitates the maintenance of sterility, gas exchange and pressure adjustment within the bioreactor vessels, tissue position adjustment within the bioreactor chambers, and tissue attachment. The bioreactor cap is transferable between the first bioreactor vessel and the second bioreactor vessel. As can be appreciated, in some forms, the first bioreactor vessel is preferably adapted to provide a static seeding chamber while the second bioreactor vessel is preferably adapted to provide a pulsatile seeding chamber, when the bioreactor vessels are used as such. In this embodiment, the cap is preferably attached to a ring stand or other frame known in the art and the bioreactor vessels are interchanged while the tissue remains affixed to the cap, via the tissue retainer, and the cap remains attached to the ring stand. In this embodiment, preferably the cap is designed with a groove that permits suspension from a ring stand/fork assembly. This mechanism of the bioreactor system allows for the maintenance of sterility of the system, simplifies single operator use and reduces the possibility of contamination of the tissue. The bioreactor cap is indirectly involved with tissue positioning within the chamber in that the elevator system is mounted to the cap. Thus, the cap must be secured to either the first or second bioreactor vessels before the tissue position may be adjusted. The cap preferably uses externally accessible filters, check valves and external resistors, so a wide range of alterations in cyclic pressures, gas outflow resistances, fluid and gas flows, and cycle timing can all be controlled without detaching the system from the bellows, actuator or opening the bioreactor vessel. Alternatively, for repetitive applications and to reduce potential operator error in settings, the cap can be configured utilizing an internal filter and a fixed air inlet/outlet cross-section tuned to a seeding protocol appropriate for the tissue type. In this configuration, desired changes in hydraulic pressure and flow waveforms (amplitude—maxima and minima rate of change), and cycle timing can be computer or operator controlled by changing the actuator timing (rate of rise, rate of descent, frequency) and stroke length.

The bioreactor system of the present invention preferably comprises a tissue retaining system or grip to hold a tissue within the bioreactor. The tissue retaining system or grip is described in U.S. patent application Ser. No. 12/481,294, the contents of which are incorporated herein by reference. The tissue retainer for securing a tubular tissue may include a retainer body defining a distal opening in communication with a proximal opening through a conduit. The retainer body may define a stepped portion and a tubular portion. In particular, the stepped portion defines a plurality of concentric steps with each of the plurality of concentric steps including a vertical wall in communication with a horizontal wall, wherein the plurality of concentric steps provides a means of custom fitting the retainer body to a particular size of tubular tissue to be retained. In one embodiment, a tissue retaining system for securing tubular tissue may include first and second tissue retainers with each of the first and second tissue retainers having a retainer body defining a distal opening in communication with a proximal opening through a conduit. The retainer body may define a stepped portion and a tubular portion. In particular, the stepped portion may define a plurality of progressively larger concentric steps (i.e. of increasing diameter) adapted to engage different sizes of tubular tissue. The tubular tissue may include opposing end portions each having an external fibrous ridge with each end portion being adapted for engagement with either the first and second tissue retainers such that fluid flow communication is established between the first and second tissue retainers through the tubular tissue.

A preferred embodiment of the bioreactor system of the present invention includes the use of two tissue retainer elements, one for securement of the proximal end of a tissue and one for securement of the distal end of the tissue. In this embodiment, the tissue retainer on the distal end of the tissue preferably includes an element complementary to the homing device optionally included in the base or distal end of the bioreactor vessel, such that the tissue retainer on the distal end of the tissue attracts and/or engages the homing device on the distal end of the bioreactor vessel providing for the tissue engaged within the tissue retainers to be centered within the bioreactor vessel. The homing device and complementary elements are preferably each a magnet, wherein the magnet in the tissue retainer and the magnet in the base or distal end of the bioreactor vessel are attracted to each other such that they form a magnetic attachment when brought into proximity with one another. The magnet attached to the tissue retainer is preferably attached to the step with the largest diameter. This magnet is preferably oriented in a circular fashion, such that it does not prevent the movement of fluid or gas through the tissue via the tissue retainer. Advantageously, the homing device prevents lateral movement during the loading of a tissue into the second bioreactor vessel. Using the valve elevator system of the present invention, tension can be applied to the tissue.

In an alternate embodiment, a plug can be placed in the distal tissue retainer preventing escape of the cell suspension during the cell seeding phase. The plug is preferably made of silicon. The plug is preferably sized to fit the tissue retainer that is part of the bioreactor system of the present invention. Since the size of the tissue retainer is determined by the type of tissue being used in the bioreactor system, the size of the silicon plug is also determined by the size of the tissue being used in the bioreactor system. Alternatively, a sleeve of material can be placed within a hollow tissue in-between the tissue retainers. Preferably, the sleeve of material is silicon. Advantageously, the sleeve can be used to inhibit fluid flow through the external holes of the second bioreactor vessel.

In alternate embodiments, the bioreactor vessel(s) and cap can be oriented in different configurations depending on the tissue being seeded. The size, shape, and orientation of the bioreactor vessel depend on the type of tissue being utilized. Additionally, the elevator screw on the bioreactor cap can be configured to attach to two or more tissue retainers where each is holding one portion of a tissue, such that a horizontal configuration or other figuration of the tissue could be utilized within the bioreactor vessel. An alternate configuration may also be necessary for tissues that do not have an internal space for the flow of fluid or gas. For example, when using skin tissue, the skin tissue is oriented in a horizontal manner, such that the tissue is parallel with the distal portion of the second bioreactor vessel and the holes though which fluid or gas is introduced into the bioreactor vessel through the distal end thereof. This allows the greatest surface area to be in contact with the fluid or gas flow. Additionally, elements of the bioreactor system, such as the plug or sleeve described above, can be used to alter the flow of liquid or gas throughout the bioreactor system, such that the flow of the liquid or gas is appropriate for the tissue in the bioreactor system. For example, all or substantially all of the holes though the distal end of the second bioreactor vessel could be plugged with the exception of those that provide fluid flow within a tubular tissue (i.e., those that are communication with the conduit through a tubular tissue), which would thereby only permit flow through the tubular portion of the tissue. Similarly, all or some of the holes that supply fluid flow through the tubular tissue could be selectively plugged or blocked while the remaining holes in the distal end of the second bioreactor vessel were left unblocked. Such a configuration would permit flow around the exterior surfaces of the tubular tissue. Of course, any combination of these is possible through selective blocking/opening of the holes. Alternatively, some or all of the holes could include one-way valves that permit fluid flow in only one direction. Further, use of the plugs, sleeves, variable restrictions, and/or one-way valves in order to restrict the flow of liquid or gas into the bioreactor vessel alters the pressures and flows not only in the chamber as a whole, but can be configured by altering specific channels between the bellows and the bioreactor chamber to be different within defined regions of the bioreactor system in relation to the geometry of the tissue or synthetic constructs to be seeded.

Any naturally derived or synthetic tissue or scaffold used for tissue engineering applications will work for purposes of the present invention. Preferably, the tissue is selected from mammalian tissue, avian tissue, or amphibian tissue. More preferably, the tissue is mammalian tissue, preferably selected from the group consisting of human, ovine, bovine, porcine, feline, canine, and combinations thereof. In a most preferred embodiment, the tissue is human tissue. The tissue to be used in the bioreactor can be any tissue suitable for use as a biological scaffold. Preferred tissues include, but are not limited to vascular tissue, organ tissue, digestive system tissue and muscle tissue, which include heart tissue, lung tissue, liver tissue, pancreas tissue, small and large intestine tissue, colon tissue, spleen tissue, gland tissue, thyroid tissue, skin, tendon, bone, and cartilage, among others. In a most preferred embodiment, the tissue is vascular tissue, preferably heart valve tissue.

The bioreactor system of the present invention preferably provides the options for static and gentle pulsatile pressure/flow environments for cell seeding and tissue conditioning, respectively. In preferred forms, the first bioreactor vessel is used for static seeding and the second bioreactor vessel is for pulsatile pressure/flow cell seeding. Advantageously, the design of the bioreactor system of the present invention offers significant advantages in terms of the creation and maintenance of a sterile environment since the tissue is preferably attached to the cap, a separate bioreactor vessel is used for static seeding and pulsatile flow, and that the entire system is a single use system.

The bioreactor system of the present invention preferably generates fluid flow both through and around the tissue scaffold. Additionally, the bioreactor system of the present invention, in one embodiment, exposes the tissue scaffold to either a liquid/gas alternating environment or an entirely gaseous environment, or an entirely liquid environment under normal operation.

Advantageously, a seeding environment is provided by the bioreactor system of the present invention that promotes cells seeding within the tissue below the basement membrane. In a preferred embodiment where the tissue is a heart valve, the first bioreactor vessel is designed to be a static seeding chamber and is designed to focus cell adhesion phase of seeding on the leaflets and at the base of the valve including the valve annulus and cuspal attachments, facilitating cell migration through the adventitia into the leaflets (i.e., seeding in addition to direct cell seeding via tissue surfaces within the lumen).

In a preferred embodiment, a bioreactor is provided according to the invention that provides a tuneable pressure, where the pressure gradient is from −5 mmHg to −20 mmHg during the pulsatile seeding phase. Preferably, a tissue is placed between two tissue retainers and connected to the bioreactor cap of the present invention. A silicon plug is placed in the bottom of the distal tissue retainer in order to prevent the escape of cell suspension within the tissue. The first bioreactor vessel, used for static seeding, was then attached to the bioreactor cap, suspending the cap and bioreactor vessel housing the tissue to the ring stand. Mesenchmyal stromal cells were suspended in 7 ml of DMEM and pipetted into the tissue using one of the ports in the cylindrical side wall of the first bioreactor vessel. The tissue was allowed to seed in the first bioreactor chamber for about 24 hours. Then, the first bioreactor vessel was detached from the bioreactor cap and the second bioreactor vessel was then attached to the cap. The silicon plug was removed from the tissue retainer and a silicon sheet with an outer diameter equal to that of the inner diameter of the tissue attached to the tissue retainers. The purpose of the silicon sheet was to inhibit fluid flow through the external holes during bellows expansion. The valve was then transferred to the second bioreactor chamber for pulsatile seeding. The valve seeding chamber was then filled with DMEM and a dedicated outflow filter was affixed to the bioreactor cap. An additional outflow/inflow with external resistance was also added. The second bioreactor chamber was then returned to the incubator and mounted to an actuator platform and a Bellows system. The actuator was activated to provide pulsatile flow within the bioreactor through the Bellows system for about 72 hours using an actuator displacement rate of 0.25 cm/min for both the up and down strokes. The pressure within the bioreactor vessel during pulsatile seeding was from about 5 mmHg to −20 mmHg. This process produced a tissue with several cells that migrated below the basement membrane of the tissue.

One of the surprising benefits of the bioreactor of the present invention is that it can be used to seed a decellularized tissue in less than 6 hours, where the tissue is ready for implant after being seeded. The bioreactor preferably takes from 4-72 hours from decellularized tissue to implant, more preferably from less than 60 to about 70 hours, more preferably from less than 50 to about 64 hours, still more preferably from less than 40 to about 60 hours, more preferably from less than 24 hours to about 56 hours, more preferably from less than 12 hours to about 48 hours, more preferably less than 8 hours to about 36 hours, and most preferably less than 6 hours.

One of the benefits of the bioreactor system of the present invention is that sterility is maintained through a combination of the geometrical relationships between the cap and seeding chambers and through the optional use of a dual o-ring seal system. In a preferred embodiment, within the bioreactor cap, a hollow cylinder extends below a bottom o-ring. The diameter of this cylinder is such that it fits freely within either the pulsatile or static seeding chambers. The diameters of both the first (static seeding) and second (pulsatile seeding) bioreactor vessels preferably bevel from a smaller diameter to a larger diameter over a short vertical height increase at the proximal ends thereof. The bottom o-ring on the bioreactor cap seals at the bottom of the bevel (smaller diameter), while the top o-ring seals at the top of the bevel (larger diameter). This provides for a “sterile zone” between the two o-rings meaning the beveled surface remains sterile. The hollow ring extending below the bottom o-ring acts a guide for assembly. This, in conjunction with the “sterile zone” concept, is functionally advantageous because it reduces the likelihood of the cap coming into contact with a non-sterile surface during assembly. To ensure that the cap remains in position and the o-ring seals against the chamber wall are maintained, the cap preferably utilizes two mid-point cantilevered fasteners that latch to the outside of either chamber. In a specifically preferred embodiment, the internal diameters of both the pulsatile and static seeding chambers bevels from 2.530 inches to 2.758 inches over a vertical height increase of 0.198 inches, however, the bevel may be any value that allows for sealing engagement of the cap. The external diameter of the hollow cylinder extending from bottom of the bioreactor cap is preferably from about 1 to 5 inches, more preferably from about 2 to 5 inches and most preferably is about 2.5 inches.

In a preferred embodiment, the bioreactor cap permits gas exchange (directly) and pressure adjustment (both directly and indirectly) through the addition of one or more, preferably at least 2, more preferably at least 3, more preferably at least 4, still more preferably at least 5, more preferably at least 6, still more preferably at least 7, and most preferably at least 8 threaded holes in the top of the cap. Conventional threaded luer ports are preferably screwed into each of the threaded holes. Thus, any combination of holes may be capped (i.e., sealed off) or left open. Additionally, any combination of external gas-sterilization filters, one-way check valves, and/or external resistors may be added. Other types of external filters and resistors may be used singularly or in combination. Gas exchange is directly accomplished via air or mixed gases flow through the holes secondary to the movement of fluid into and out of the bioreactor chamber. Note that to maintain sterility, all inlet gasses entering the chamber are preferably sterilized first (i.e., must pass through filters). For a given actuator displacement rate, the pulsatile pressure may be adjusted through the addition/subtraction of external filters or one-way check valves or through the adjustment of 1 or more external resistors.

Advantageously, this embodiment of the bioreactor system provides for a high degree of pressure adjustment. Preferably, the cyclic pressure induced in the environment where the decellularized tissue is recellularized does not disrupt or put damaging levels of stress on the cells therein. Even more preferably, the cyclic pressure ranges from about 0.5 mmHg to 200 mmHg, more preferably, from about 1 mmHg to 150 mmHg, still more preferably, from about 1.5 mmHg to 100 mmHg, more preferably, from about 2 mmHg to 50 mmHg, even more preferably, from −2.5 mmHg to 30 mmHg, still more preferably from 2.6 mmHg to 25 mmHg, even more preferably from 2.7 mmHg to 20 mmHg, still more preferably from 2.8 mmHg to 15 mmHg, even more preferably from 2.9 mmHg to 12 mmHg, and most preferably, from −3 mmHg to 10 mmHg. The preferred range for cyclic pressure is one that does not disrupt the cells, but does promote cell metabolism and various desirable cell activities in any combination, (including proliferation, phenotype differentiation, protein synthesis, cell migration, cell signaling, cell homeostasis), encourages subsurface migration, into the tissue scaffold matrix or physically moves cells subsurface via pressure differentials creating vacuum or suction, or with positive and negative pressure gradients, or with alternating maximum and minimum positive pressures on and across tissue layers.

In a preferred embodiment where the first bioreactor vessel is used for static cell seeding in a heart valve, the first bioreactor vessel is preferably made of an injection molded polymer (e.g. polystyrene) component used during the initial seeding of valves with autologous bone marrow. This chamber is specifically designed to focus seeding at the valve annulus, increase the probability of cell attachment to the valve scaffold, permit measurement of biological and operational parameters, and to maintain sterility of the system. The first bioreactor vessel or static seeding chamber is designed with a conical bottom. The minimum diameter preferably occurs at the bottommost end of the chamber.

In a preferred embodiment, any one of the ports present on the second bioreactor vessel can be used as a way to monitor the biological and operational parameters. Any device for measuring or monitoring biological or operational parameters can be used in connection with the second bioreactor vessel of the present invention. In a preferred embodiment, standard female luer connectors molded along the vertical height of the chamber permit monitoring of biological and operation parameters, including but not limited to, pressure, pH, pO₂ and pCO₂, energy or protein synthesis metabolites (e.g., lactate, glucose, cleavage proteins, soluble proteins, etc.) using standard clinical equipment. As is known in the art, each of these can provide an indication of biological activity occurring in the vessel. For example, the monitoring of the pH and/or metabolites in the system gives an indication of the growth and functionality of cells within the tissue. Advantageously, these ports can also be used for aseptic media exchanges, additions, or removals; additions of signaling or therapeutic proteins or small molecules, drugs and metabolic enhancers and nutrients.

The bioreactor assembly is preferably designed to generate a uniform pressure within the pulsatile seeding chamber, exposing both the inside and outside of the tissue to the same pressure loading conditions. In a preferred embodiment, an external actuator is used such that when the bellows is compressed, conditioning and nutrient media is driven into the second bioreactor from the compressible vessel used for pulsatile motion. The magnitude of the pressure generated is determined partially by the total air outflow resistance generated by the bioreactor cap but also partially by the rate of media flow into the pulsatile seeding chamber or second bioreactor vessel. The rate and extent of compression and the rate and extent of media flow into the pulsatile chamber are directly related and are both tunable. Thus, for a given total gas outflow resistance, increased compression rates result in increased chamber pressures. In a preferred embodiment, media flow through the valve scaffold is unrestricted. Thus, both the inside and outside of the scaffold are exposed to the same pressures, resulting in the absence of physiologic conduit pulsation while compressing the tissues and thus stressing or deforming the cells by transmitting hydraulic forces to the cells and matrix. Preferably, the baseplate or distal end of the second bioreactor vessel is designed such that fluid flows both through the outside and inside of the tissue, preferably a heart valve. Media flow through the valve is facilitated by a through hole centrally located on the baseplate. Media flow outside the valve is achieved through concentric rows of perforations in the baseplate. Advantageously, this type of configuration provides for operator control of the spatial distribution of hydraulic resistances and flows which can preferably be configured to reduce shear inside or outside the conduit, thus avoiding stripping of seeded cells from the surface of the tissue. The configuration and diameters of the holes can be altered for any desired ratio to result in specific quantitative levels of differential flows and pressures across and parallel to the tissue planes. It can be configured so 100% of the flow is inside or outside a vessel structure (or any ratio in between). Flow through tubular structures simply overflows at the top, thus continuously returning the chamber and reservoir without external flow loops.

The bioreactor system of the present invention preferably provides for an environment that allows a pilot cell population to more easily or readily migrate into the tissue below the basement membrane. It was surprisingly found that this pilot population of cells leads to greater repopulation of cells in the tissue once it is implanted into the intended recipient. This is because it has been surprisingly found that the pilot cell population attracts other cells into the tissue matrix after the tissue is implanted into the recipient. The bioreactor system of the present invention uses a static seeding phase and a phase using pulsatile motion, where the pulsatile motion preferably comprises a repeated cycle of fluid entering the second bioreactor vessel and exiting the bioreactor vessel. Unlike previous bioreactors that have tried to mimic in vivo conditions for cells seeding, it was surprisingly found that pulsatile focus generating conditions that do not mimic in vivo conditions, such as that provided by the bioreactor system of the present invention, leads to seeding of cells further into the tissue construct, providing a pilot population of cells that attract more cells into the tissue when the tissue is implanted into the recipient.

In a most preferred embodiment, a tissue, preferably a heart valve, is harvested and decellularized prior to being placed in the bioreactor system of the present invention. The tissue may be decellularized according to any protocol known in the art, but is preferably decellularized according to U.S. patent application Ser. No. 12/813,487, the contents of which are incorporated herein by reference. The tissue is then secured between two of the tissue retainers, with one being on the proximal end of the tissue and one being on the distal end of the tissue. The tissue retainer on the proximal end of the tissue is then connected to the elevator mechanism on the cap of the bioreactor, where the cap of the bioreactor is attached to a ring stand or similar mechanism. The first bioreactor chamber is then secured to the cap, where the double O-rings in the cap engage the two beveled edges of the first bioreactor chamber and the clamps on the cap are secured to the bioreactor vessel. The first bioreactor vessel may already include cells and/or a cell matrix to be seeded onto the tissue or the cells may be added to the first bioreactor vessel using one of the ports present on the cylindrical wall of the first bioreactor vessel. After the static seeding phase has ended, the first bioreactor vessel is removed from the cap and affixed tissue and the second bioreactor vessel is attached to the bioreactor cap, such that the tissue is now inside of the second bioreactor vessel. The elevator mechanism is used to move the tissue and attached tissue retainer such that the magnet present within the distal tissue retainer attracts the magnet present in the distal end or bottom of the second bioreactor vessel. This allows the tissue to be centered within the second bioreactor vessel. The tension on the tissue is then adjusted to the desired tension. The cap and second bioreactor assembly is then removed from the ring stand and coupled to a system, such as the Bellows System, that allows for fluid or gas to be pushed into the bioreactor vessel and removed from the bioreactor vessel. Preferably, a mechanism that has the ability to monitor and/or control the biological and mechanical properties of the bioreactor is coupled to the second bioreactor vessel. This mechanism is used to carry out pulsatile conditioning on the tissue. At the end of the pulsatile phase, the tissue is then removed and implanted into the recipient. The bioreactor vessels and cap are then discarded.

Preferably, patient specific cells are utilized in the reseeding process, depending on the type of tissue utilized with the bioreactor system of the present invention. In a preferred embodiment, the cell source is 1) patient-specific, 2) easily accessible in a clinical setting and 3) requires minimal processing prior to tissue seeding. In an embodiment where a heart valve is used, cells utilized for reseeding preferably include, but are not limited to endothelial cells, myofibroblasts, mesenchymal stem cells, and combinations thereof.

### BRIEF DESCRIPTION OF FIGURES

FIG. 1 is a front perspective view (A) and a side view of the first bioreactor vessel (B);

FIG. 2 is a front perspective view (A), a bottom plan view (B), and a side view of the second bioreactor vessel (C);

FIG. 3 is a side view (A), a bottom plan view of the bioreactor cap (B), and a front perspective view (C);

FIG. 4 is a front perspective view (A), a side view (B), a bottom plan view (C), and an additional side view (D) of the housing for the elevator mechanism used in connection with the bioreactor cap;

FIG. 5 is a front perspective view (A) and a side view (B) of the elevator mechanism used in connection with the housing and bioreactor cap;

FIG. 6 is a front perspective (A) and a side view of the bellows (B);

FIG. 7 is a top plan view (A), a side view (B), a bottom plan view (C), and a top plan view (D) of the homing device using in combination with the tissue retainer;

FIG. 8 is front perspective view (A), a top plan view (B), a side view (C), and a bottom plan view (D) of the ring magnet holder;

FIG. 9 is a side perspective view (A), a side view (B), and an additional side perspective view (C) of the threaded ring;

FIG. 10 is an exploded view of an entire bioreactor system;

FIG. 11 is an additional exploded view of a bioreactor system;

FIG. 12 is a further exploded view of a bioreactor system without the housing;

FIG. 13 is a side view of the bioreactor system using the second bioreactor vessel;

FIG. 14 is a side view of the bioreactor system using the first bioreactor vessel;

FIG. 15 is a graphical representation of the pressure profile observed in Example 2;

FIG. 16 is a set of photographs of H&E staining for ovine pulmonary valve leaflets observed in Example 2 (A and B at 100×, C &D at 200×);

FIG. 17 is a front perspective view (A), a bottom plan view of the base plate cage (B), and a side view (C);

FIG. 18 is a perspective view (A), a bottom plan view (B), and a side view (C) of the flapper;

FIG. 19 is a side view of the tissue cage and base plate used together; and

FIG. 20 is a side view (A) and bottom plan view (B) of the base plate;

FIG. 21 is top perspective view (A), a top plan view (B), and a side view (C) of the ring magnet; and

FIG. 22 is a side view of the bioreactor system of the present invention with a tissue in the bioreactor system.

### DETAILED DESCRIPTION

An innovative bioreactor system is provided herein. The bioreactor of the present invention addresses many potential regulatory concerns, while providing the functionality necessary to generate a subsurface cell population within the leaflets of decellularized heart valves. The system and methods of the present invention optimizes the multifaceted aspect of valve seeding to establish the best possible pilot population of viable cells within the PVL (pulmonary valve leaflets).

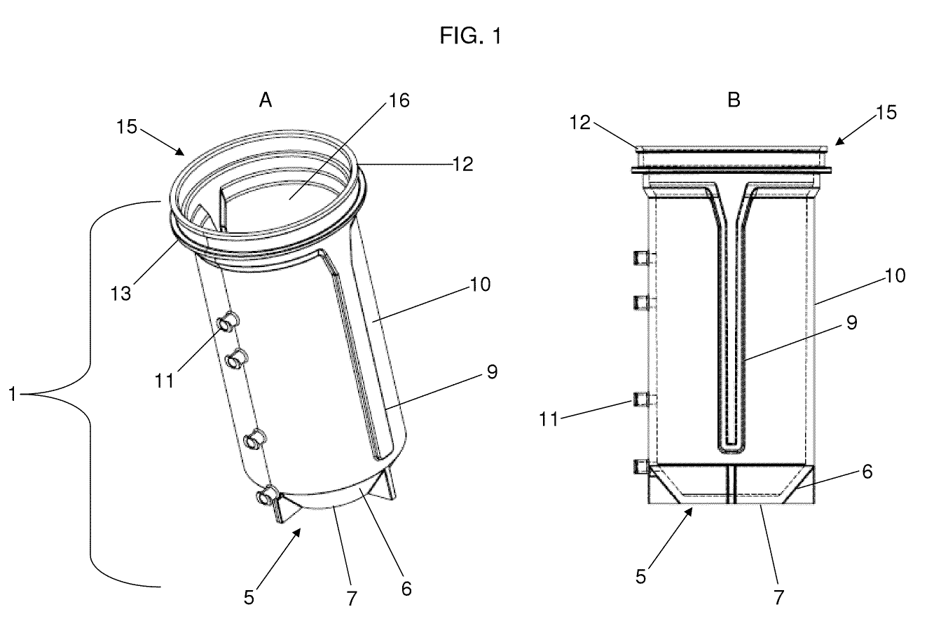

FIG. 1 (A and B) illustrates the first bioreactor vessel 1 that has two opposed ends 5, 15 and a continuous cylindrical side wall 10 extending therebetween. The distal end 5 of the first bioreactor vessel is preferably closed, where the closed end preferably gradually narrows 6 in diameter on an angle in a frustum conical shape away from the cylindrical sidewall 10 to a flat plane 7 having a surface area less than that of the opening at the other, proximal end 15 of the first bioreactor vessel. It is preferred that the closed end of the first bioreactor vessel provides a narrowed bottom portion allowing any fluid material within the retainer body to concentrate at the bottom or at the distal end. The bioreactor vessel 1 has one or more ports 11 on the continuous cylindrical side wall 10. Preferably, the first bioreactor vessel has a beveled edge 12 along the opening 16 on the proximal end 15 such that the beveled edge 12 can engage a cap, attachably sealing the cap and bioreactor vessel 1. In the embodiment presented in FIG. 1, the bioreactor vessel 1 comprises a stepped portion 13 on the proximal end 15, such that there are two beveled edges 12 that have the ability to engage the bioreactor cap forming a seal. The first bioreactor vessel additionally has at least one groove 9 along the cylindrical sidewall allowing for the upward and downward movement of an elevator mechanism.

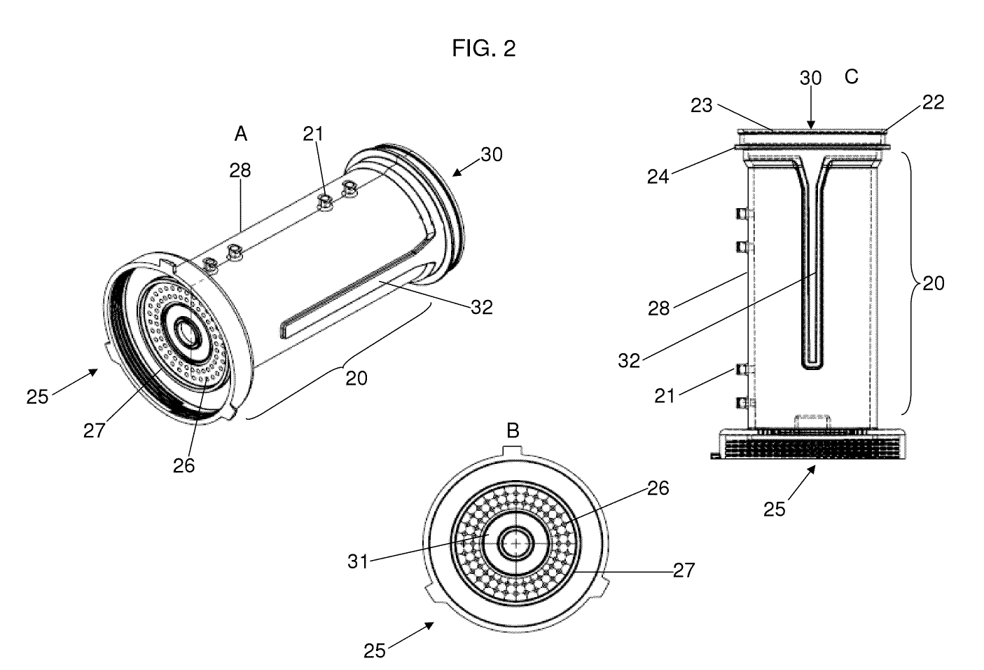

FIG. 2 (A, B & C) illustrates the second bioreactor vessel 20 having a distal end 25, a proximal end 30, and a continuous cylindrical side wall 28 therebetween. The distal end 25 has a plurality of openings 26 forming a ring on the bottom surface 27 of the distal end 25. The openings 26 on the distal end 25 of the second bioreactor vessel 20 preferably provide access for liquid or gas to enter and exit the bioreactor system. The second bioreactor vessel 20 has one or more ports 21 on the continuous cylindrical side wall 28. Preferably, the second bioreactor vessel 20 comprises a homing device 31 (shown in FIG. 2B) in the base or distal end 25 of the second bioreactor vessel 20. This homing device 31 preferably attracts a counterpart in the base of a tissue retainer or grip, such that the grip is positioned in the center of the surface area of the base or distal end of the bioreactor vessel when the grip is placed in contact with the distal end of the bioreactor vessel. The second bioreactor vessel 20 has a beveled edge 22 (FIG. 2C) along the opening 23 on the proximal end 30 such that the beveled edge 22 engages the bioreactor cap, attachably sealing the cap and bioreactor vessel 20. In the illustration, the second bioreactor vessel 20 comprises a stepped portion forming a second beveled edge 24 on the proximal end 30, such that there are two beveled edges 22, 24 that engage the bioreactor cap forming a sterile seal. The second bioreactor vessel additionally has at least one groove 32 allowing for the upward and downward movement of the elevator mechanism.

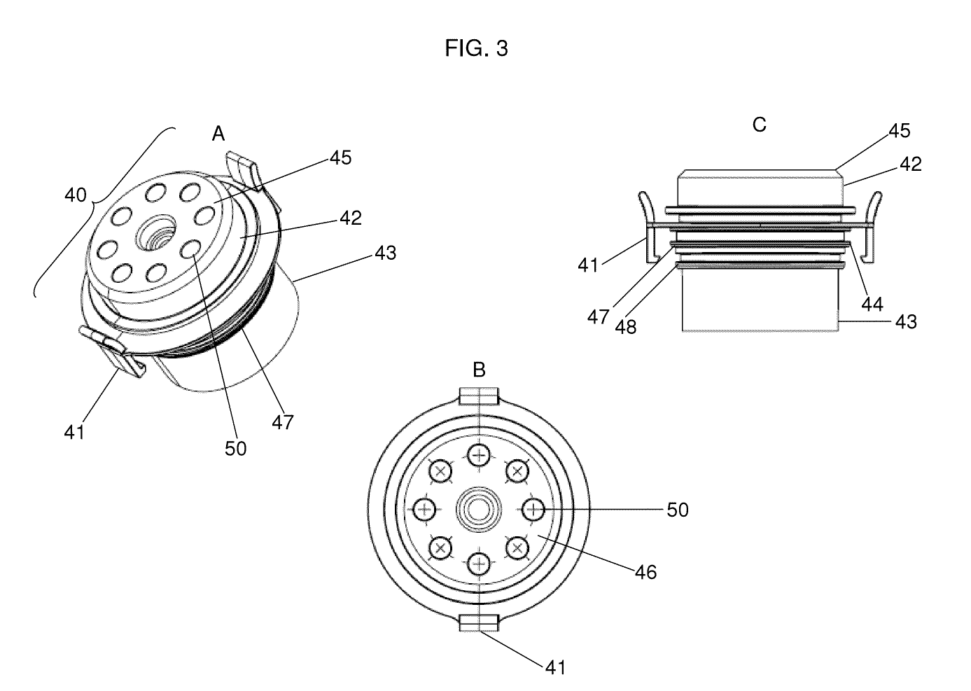

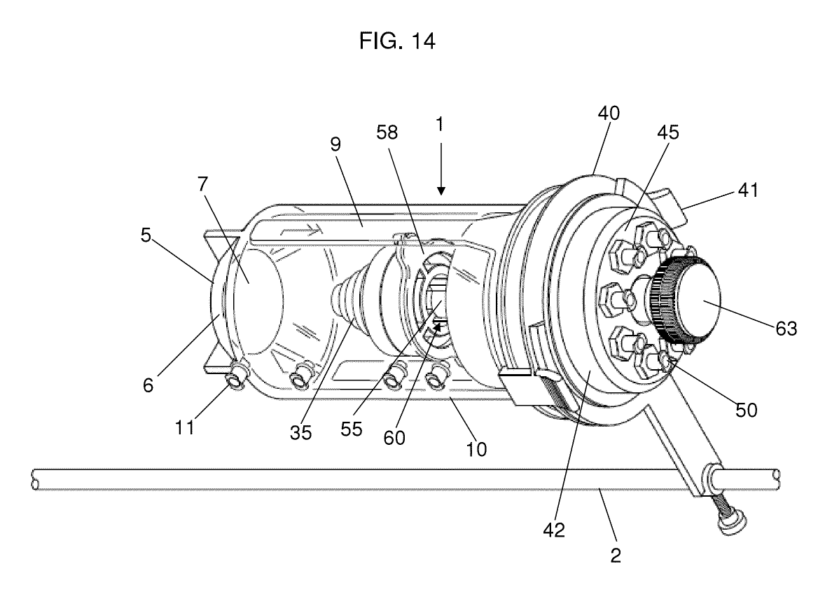

FIG. 3 (A, B, & C) illustrates the cap 40 of the bioreactor system of the present invention. The cap 40 preferably detachably connects to each of the bioreactor vessels 1, 20. The cap 40 has an overall surface area that is large enough to cover the opening of the bioreactor vessel for which the cap is being used in connection with. The cap has a top face 45 which may have one or more holes or ports 50. Preferably, these one or more holes or ports 50 allow for the escape of air or gas within the bioreactor vessel, or allow a user to alter the internal pressure of the bioreactor vessel. A plug, resistor, or filter may be used to block the entrance or exit of gas or other materials from each hole or port 50 in the cap 40. The cap 40 preferably has one or more fasteners, clamps, or other attachment mechanisms 41 allowing the cap 40 to detachably connect to the bioreactor vessel 1, 20. In this embodiment, the cap 40 has a stepped portion 42 that includes an end with a smaller diameter than that of the overall diameter of the cap 40. A cylindrical side wall 43 extends from the bottom face 46 where a step 47 is created between the bottom face 46 of the cap and the cylindrical side wall 43. The step or edge 47 of the cylindrical side wall 43 comprises a ring of material 44 that engages the bioreactor vessel in such a way to provide a seal when the cap 40 is affixed to the bioreactor vessel. In the depicted embodiment, a second ring of material 48 is attached to the step or edge 47 so that the material engages the bioreactor vessel in such a way as to provide a seal when the cap 40 is affixed to the bioreactor vessel. The material preferably engages the beveled portions of the bioreactor vessel to form a seal. This embodiment, comprising a double seal between the bioreactor chamber and the cap ensures a sterile environment within the bioreactor vessel and illustrates an advantage over bioreactors in the prior art. Additionally, threaded leur fittings 120 (shown in FIG. 14) can be attached to the holes 50 in the cap 40 prior to the tissue attaching to the cap. The threaded leur fittings allow a user to cap the holes or attach filter, restrictors, and mechanisms to monitor the internal environment of the bioreactor vessel.

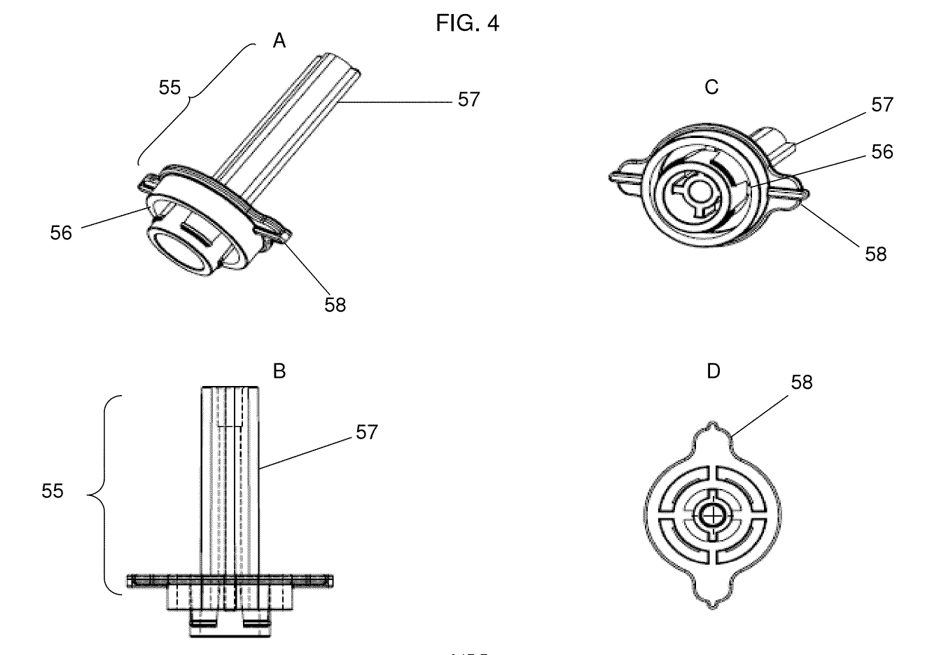

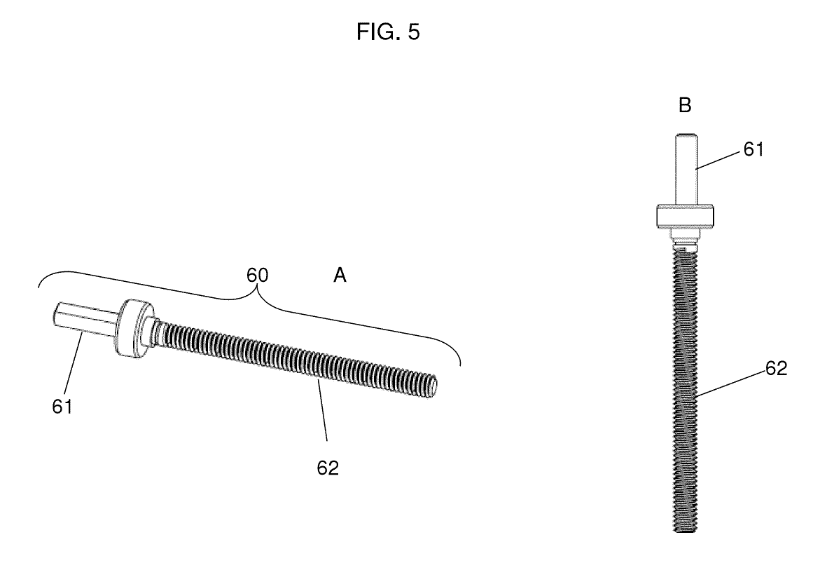

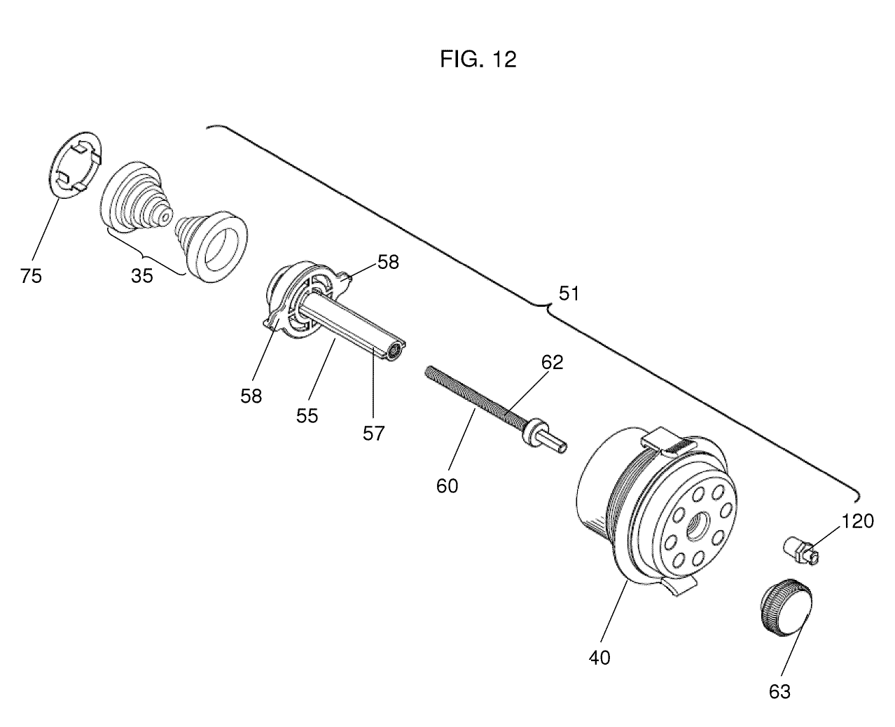

FIG. 4 (A, B, C, and D) illustrates the housing 55 for the elevator mechanism for the bioreactor cap 40. The internal elevator mechanism is attached perpendicularly to the bottom surface area 46 of the cap 40. The elevator mechanism can be any known in the art, but is preferably a screw mechanism 60 as shown in FIG. 5, operably engaged with the housing 55. The elevator mechanism 60 preferably has a threaded mechanism 62 allowing the upward and downward movement of the tissue attached to the elevator mechanism 60. The elevator mechanism is preferably affixed to the cap through a hole 50 (preferably the middle hole) in the top face 45 of the cap 40 as shown in FIG. 3, where the elevator mechanism or screw 60 has a portion that extends through 61 the top face 45 of the cap 40 and into the bioreactor vessel when the cap 40 is affixed to the open end of the bioreactor vessel, such that part of the elevator mechanism 60, the portion that extends 61 through the top face 45 of the cap 40 is outside of the bioreactor vessel and part of the elevator mechanism, the housing 55, is inside the bioreactor vessel. An attachment element 56 is also provided that is sized to secure attachment of the tissue retainer 35. The elevator mechanism 60 preferably engages an element, such as a knob 63 as shown in FIG. 12, allowing the upward or downward motion of the elevator mechanism 60 through rotation of the knob 63. The elevator housing 55 also includes bell shaped tabs 58 for attaching the elevator housing 55 including the elevator mechanism 60 to the bottom face 46 of the cap 40. In a preferred embodiment, an outwardly extending groove 57 is present at some point along the length of the elevator housing 55 that allows engagement with the groove 9 (FIG. 1) and for attachment of an o-ring. The o-ring is used to engage the top face 45 of the cap 40 to form a seal. The outwardly extending groove 57 of the elevator housing 55 engages a groove 9, 32 in the bioreactor vessels 1, 20 allowing for limited side to side movement of the elevator mechanism 60 during the upward and downward movement thereof. The element for activating and allowing motion of the elevator mechanism 60 is preferably attached to the top face 45 of the cap 40, such that a user can control the upward or downward motion of the elevator mechanism 60 while the cap 40 is attached to the bioreactor vessel 1, 20. This element may be any element that allows for a user to control the upward or downward movement of the elevator mechanism, but is preferably a rotatable knob. The housing 55 for the elevator mechanism 60 surrounds the elevator mechanism 60 without contacting the elevator mechanism 60 directly, such that the elevator mechanism 60 may be allowed upward and downward motion within the housing 55.

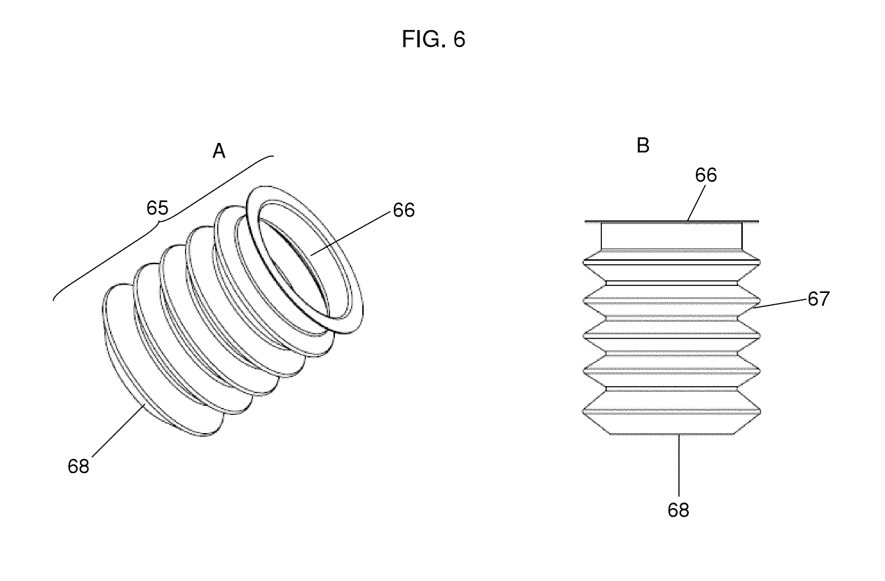

In an alternate embodiment, the second bioreactor vessel 20 is used in combination with a Bellows system (Bellows Systems, Ventura, Calif.). The bellows 65 is shown in FIG. 6 (A and B) and includes a blow molded polymer component used to drive conditioning media into and out of the second bioreactor vessel 20 for pulsatile conditioning of a tissue. The opening 66 of the bellows 65 is used to secure the proximal end of the bellows 65 to the distal end 25 of the second bioreactor vessel 20. The distal end 68 of the bellows 65 is closed. The bellows 65 has accordion-like steps 67 which compress and expand allowing fluid to migrate from inside the bellows 65 into the second bioreactor vessel 20.

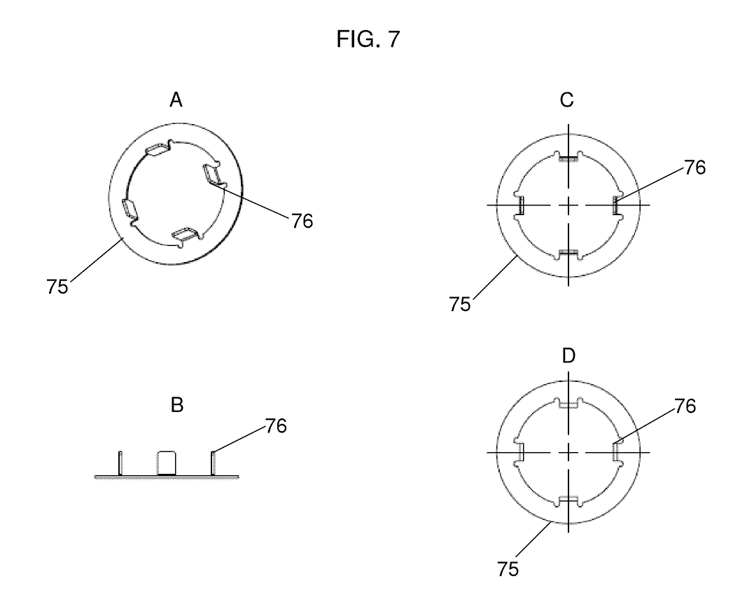

FIG. 7 (A, B, C, and D) illustrates one embodiment of the homing device 75 which is attached to the distal end of one of the tissue retainers 35 of FIG. 12. The homing device, preferably a magnet 75 is attached when the upward protrusions 76 contact and frictionally engage the inner surface area of the tissue retainer 35.

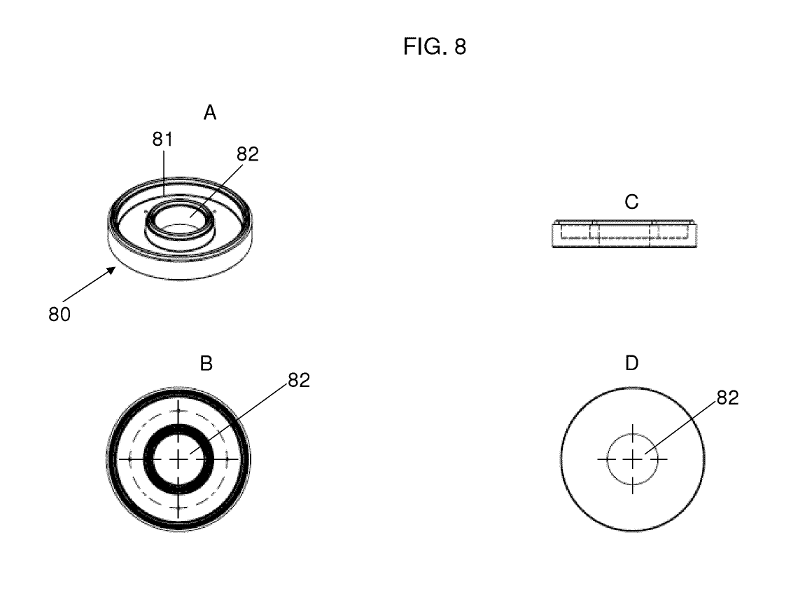

FIG. 8 (A, B, C, and D) illustrates a holder 80 for the homing device or magnet 75. The holder 80 can be attached to the distal end of the tissue retainer 35 after the homing device 75 is attached to the tissue retainer 35. The holder 80 has a circular channel or indentation 81 for receiving the homing device 75. Additionally, the holder 80 has a circular hole 82 in the center for the movement of fluid.

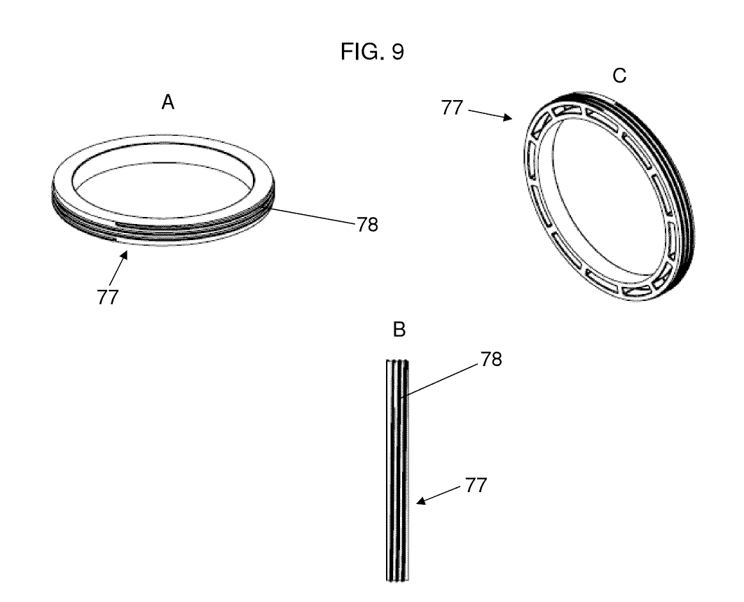

FIG. 9 (A, B, and C) illustrates a threaded ring 77 for attaching the second bioreactor vessel 20 to the bellows system 65. The threaded ring 77 preferably has helical protrusions 78 around the threaded ring 77 allowing a secure attachment to the bellows system 65.

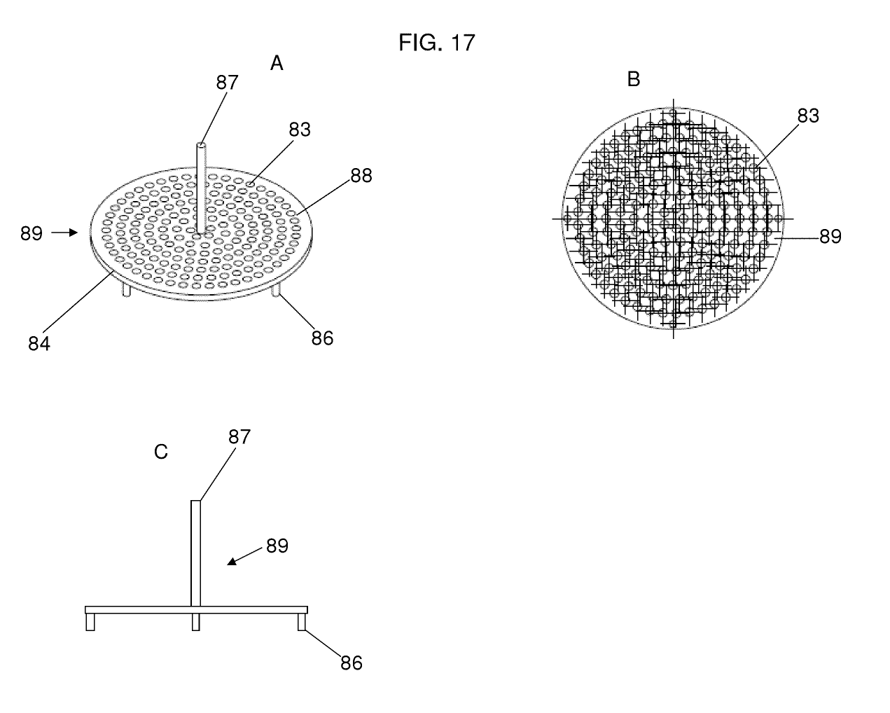

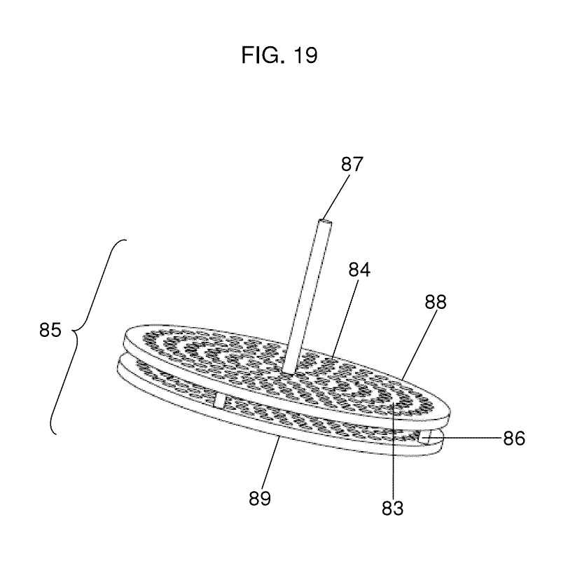

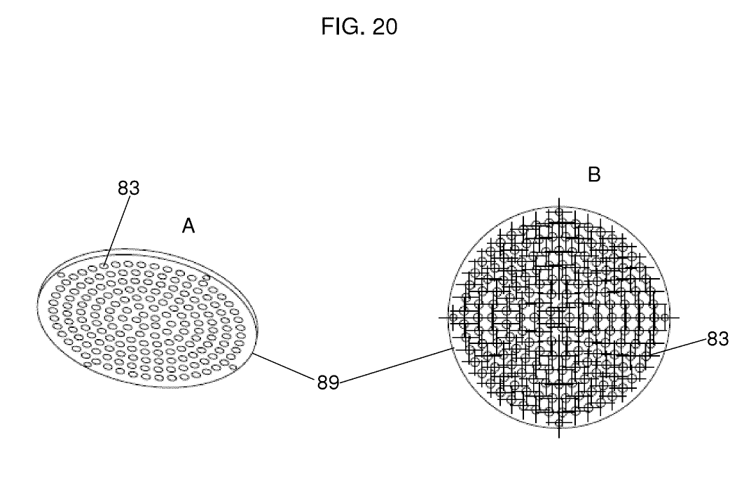

In an alternate embodiment, the bioreactor can be configured to accommodate many types of tissues, depending on the size and shape of the tissue. As an example, where a flat piece of tissue is used, a tissue cage 85, as shown in FIGS. 17 (A, B, and C) 19, and 20 (A and B) can be used. In this embodiment, a base plate 89 (shown in FIGS. 17 and 20) with holes 83 is centered inside of the second bioreactor vessel 20 at the distal end 25 such that the top face of the base plate 89 is facing the proximal opening 23 of the second bioreactor vessel 23. Next, a piece of tissue, preferably, a flat piece of tissue, is then placed on the base plate 89. The top piece 84 of the tissue cage 85 is then placed over the tissue such that the feet 86 of the top piece 84 contact the base plate 89. The top piece 84 is positioned using the rod 87 extending from the top face 88 of the top piece 84. An illustration of the interaction between the base plate 89 and top piece 84 is shown in FIG. 19, where the top piece 84 of the tissue cage 85 is on top of the base plate 89. In use, the tissue would be between the base plate 89 and the top piece 84, creating a tissue cage 85 as shown in FIG. 19.

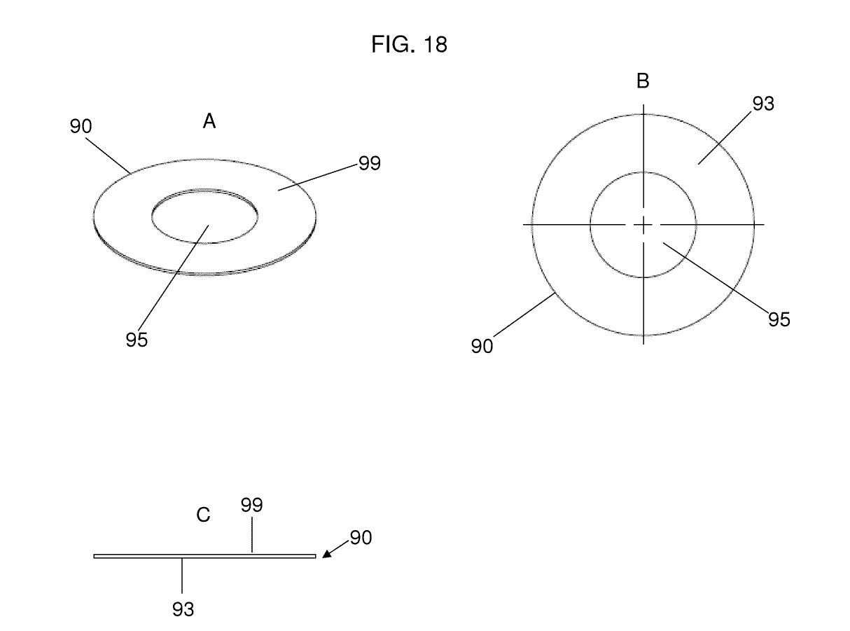

FIG. 18 illustrates a flange 90 that can be used in the second bioreactor vessel 20 in order to restrict the flow of liquid or gas from the bellows system 65. The flange 90 has a top face 99 and a bottom face 93. There is a circular opening 95 in the center of the flange 90 for fluid to move through.

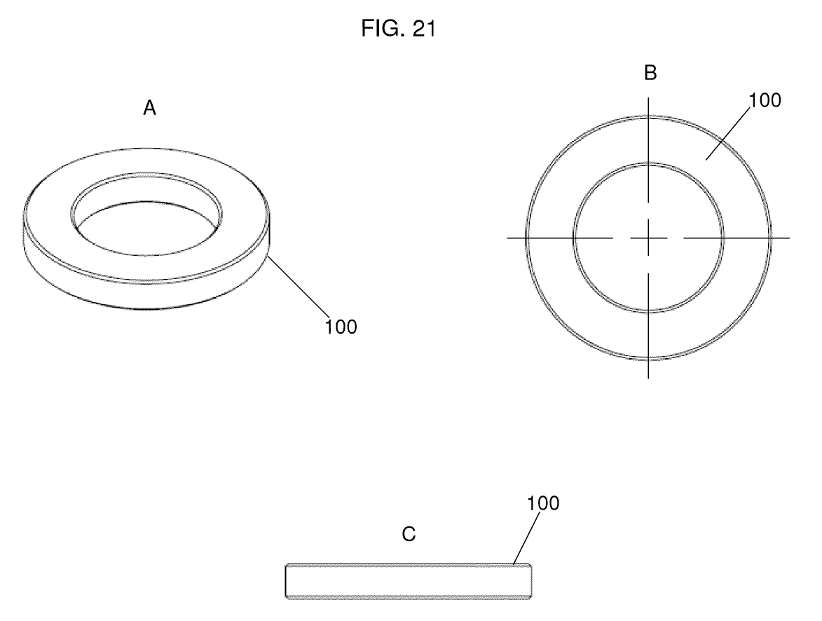

In an alternative embodiment, the base plate 89 illustrated in FIG. 20 completely replaces the element with the holes 26 on the distal end 25 of the second bioreactor vessel 20 as shown FIG. 2. FIG. 21 depicts an embodiment where the homing device 75 is a ring magnet 100.

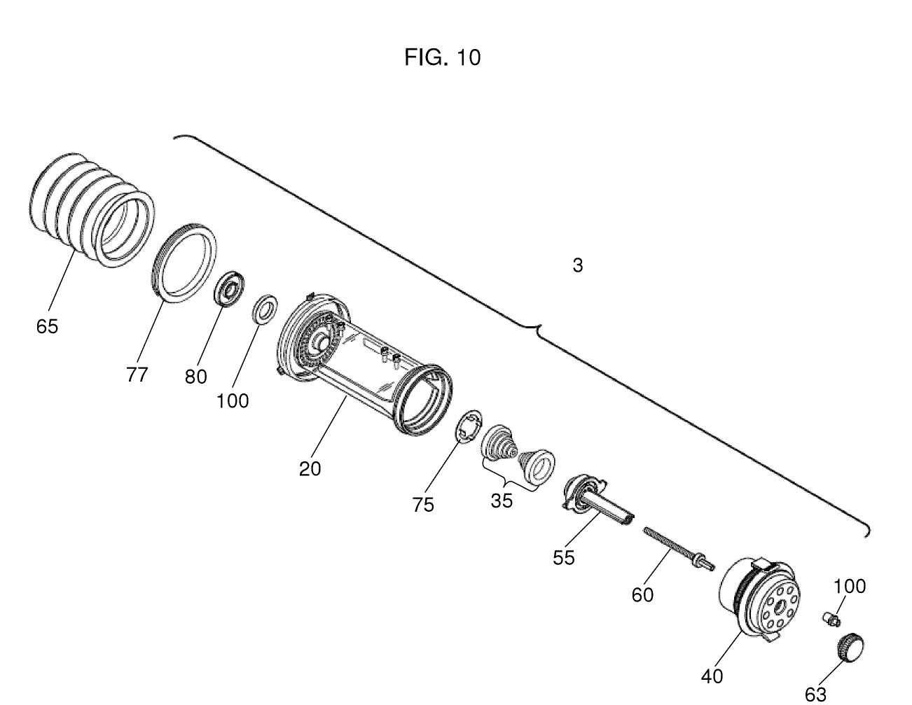

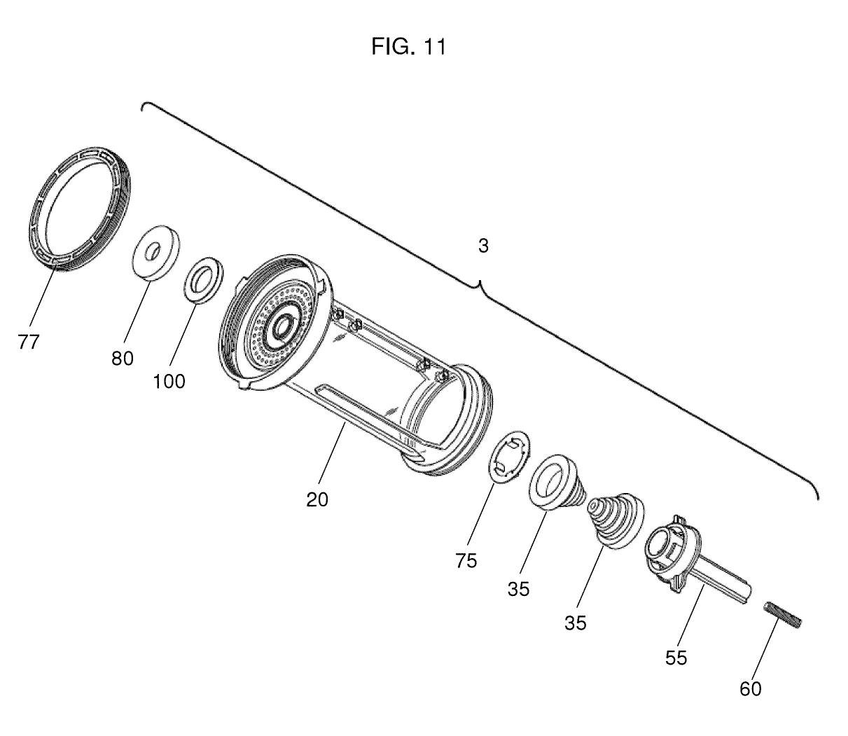

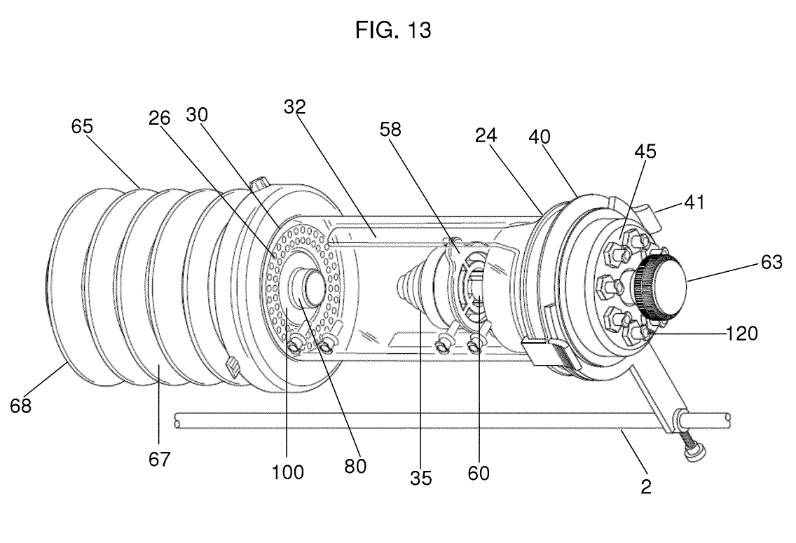

FIGS. 10, 11, and 12 provide exploded versions and FIG. 13 provides an assembled view of the bioreactor system for pulsatile seeding 3 utilizing the second bioreactor vessel 20. The bellows 65 connects to the threaded ring 77, which attaches the ring magnet holder 80 and the ring magnet 100 to the distal end of the second bioreactor vessel 20. The homing device 75 attaches to the bottom of the first tissue retainer 35 and the second tissue retainer 35 attaches to the elevator mechanism housing 55, which houses the elevator mechanism 60. The elevator housing 55 and elevator mechanism 60 extend through the cap 40 and attach to a knob 63, which allows for the upward and downward motion of the elevator mechanism thereby moving the tissue. FIG. 13 provides for an assembled version of the bioreactor system utilizing the second bioreactor vessel, where the cap 40 is attached to a ring stand 2. In a most preferred embodiment, the cap 40 remains attached to the ring stand 2 (shown in FIGS. 13 and 14) throughout the process of conditioning and seeding the tissue. The tissue remains attached to the cap 40 throughout the process also.

FIG. 12 provides for the elevator mechanism system 51, which preferably comprises the elevator mechanism housing 55, the elevator mechanism 60, one or more tissue retainers 35, and an element or knob 63 for moving the elevator mechanism system 51 up and down. The elevator mechanism 60 preferably has a threaded construction 62, such as a screw. The elevator mechanism housing 55 provides for attachment tabs 58 for attaching the housing 55 to the tissue retainer 35. The elevator mechanism 60 is housed in the elevator mechanism housing 55 and is placed through the bioreactor cap 40 where the housing 55 attaches to the cap 40 and the elevator mechanism attaches to an element or knob 63 providing upward or downward motion of the elevator mechanism. There is a groove 57 on the housing 55 that engages a similar groove on either bioreactor vessel for facilitating the upward and downward movement of the elevator mechanism system 51. The cap 40, does not move, but stays in place as the elevator mechanism system 51 moves up and down within the bioreactor vessel.

FIG. 14 provides for the bioreactor system for static seeding which comprises the first bioreactor vessel 1, the cap 40, and the elevator mechanism 60 with housing 55. The cap is attached to a ring stand 2 in a preferred embodiment. The tissue is preferably connected to the end of the tissue retainer 35, which is connected to the elevator mechanism 60. The knob 63 on the top face 45 of the cap 40 allows for the upward and downward motion of the tissue so that the tissue can contact or nearly contact the bottom 7 of the first bioreactor vessel 1. Preferably, the bottom 7 of the first bioreactor vessel 1 has cells for seeding into the tissue.

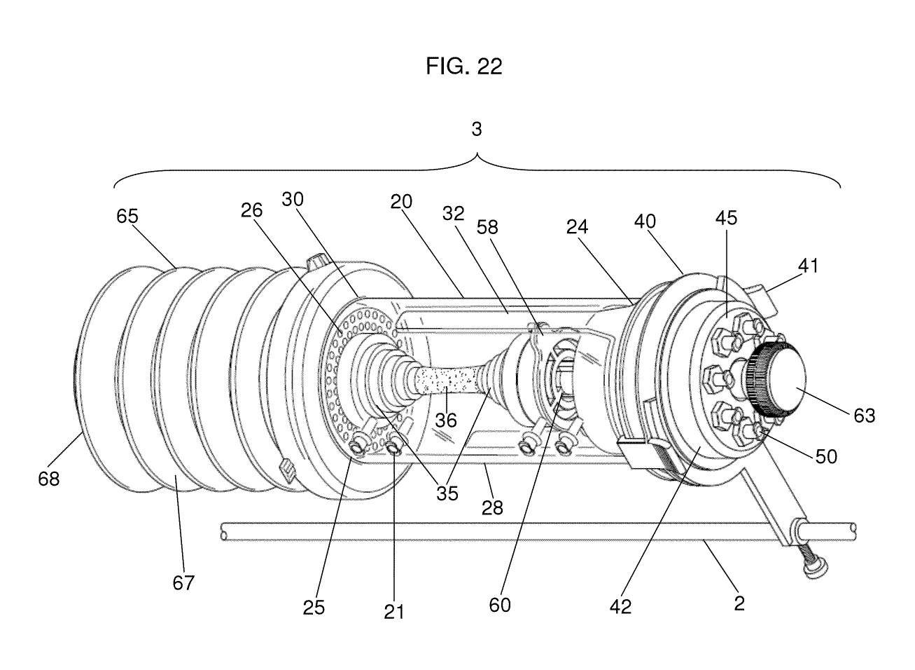

FIG. 22, provides for the bioreactor system for pulsatile seeding 3. This embodiment employs two tissue retainers 35 and the tissue 36 placed therebetween within the second bioreactor vessel 20. In a preferred embodiment, fluid would flow up into the second bioreactor vessel 20 from the bellows 65 when the accordion like steps 67 compress and expand. The fluid enters the second bioreactor chamber through the openings 26 in the distal end 30 of the second bioreactor chamber. The fluid flows in and out of the second bioreactor chamber 20 until the seeding is complete.

### EXAMPLES

The following non-limiting examples are included to illustrate the invention.

### Example 1

The bioreactor system was used to accomplish two global objects, including 1) the establishment of a pilot cell population in previously decellularized, collagen-conditioned valve allograph scaffolds and 2) the pre-implantation, pulsatile conditioning of valve scaffolds using optimized media formulations. These objectives were accomplished in two distinct phases (i.e., static and pulsatile seeding), in which each phase utilized a functionally optimized, disposable chamber.

### Materials and Methods

Initial Valve Attachment: The distal end (great vessel) of a heart valve was first attached using sutures or surgical staples to the tissue retainer or grip adaptor. The grip adapter was then attached to the valve elevator. The grip adapter was secured to the elevator by an interference fit. These processes were performed in biosafety cabinet.

1) Establishment of a pilot cell population—static seeding: The cap/valve assembly was inserted into the static seeding chamber, and the cap was latched in place. The operator then adjusted the vertical position of the valve such that the proximal end of the valve (valve annulus) was suspended in the conical portion of the seeding chamber. As described above, positioning of the valve within this section of decreasing cross-sectional area was performed in an effort to focus cell seeding at the valve annulus. Cell culture media was then added to the chamber through access ports along the vertical length of the chamber, to a level at or just above distal attachment point. Bone marrow or other cell suspensions were injected into the conical section of the chamber (near valve annulus) through the bottom access port. The closed assembly was then transferred from the biosafety cabinet to an incubator at 37° C. The chamber remained in the incubator for a pre-determined static dwell period (ideally 24-48 h). Following the dwell period, the seeding chamber was transferred from the incubator to the biosafety cabinet and cap/valve assemble was removed from the static seeding chamber. As the valve was removed, the bone marrow and cell culture media remained in the seeding chamber, and the chamber was discarded accordingly.

2) A second CMH grip adapter was sutured or stapled to the proximal end of the valve, and a magnetic, stainless steel ring was secured to the bottom of the second grip adapter. The cap/valve assembly was then inserted into the pulsatile seeding chamber, and the cap was latched in place. The operator then lowered the vertical position of the heart valve using the valve elevator assembly until the proximal grip adapter engaged the magnetic retention ring on the base plate of the pulsatile chamber. The operator could then adjust the tension in the valve conduit to the desired level. Cell culture medium was then added to the chamber through chamber access ports.

The assembled bioreactor was then transferred to an incubator and mounted onto an actuator stage (Bellows Systems, Ventura, Calif.). Following implementation of the desired bellows compression rate, chamber pressure was adjusted through the addition/subtraction of external filters and through the addition/subtraction of outflow resistance using an external resistor. The valve scaffold was then conditioned under pulsatile loading conditions for a pre-determined period (ideally 1-336 h). During pulsatile loading, the entire seeding chamber experienced a uniform pressure. That is, there was no pressure differential between the inside and outside of the valve scaffold. Thus, the valve conduit did not exhibit the pulsation observed under physiologic loading conditions.

During bellows compression and transfer of fluid from the bellows to the seeding chamber, pressure built within the chamber, reaching a maximum at the point of total bellows compression. Typical seeding protocols ideally involve a predetermined dwell period under full bellows compression (seeding chamber full). Chamber pressure decays throughout this dwell period until equalizing with the ambient pressure (outside chamber). Following the dwell period, bellows compression was released. During expansion, a slight vacuum was created within the seeding chamber and air is drawn in through the external filters. The valve scaffold was exposed to sterilized air as the cell culture media retreated to the expanding bellows. Upon full bellows expansion, the valve scaffold was entirely exposed to a gaseous environment. Pulsatile loading cycles were repeated as some capacity over the entire pulsatile seeding period. Physiologic parameters were monitored throughout this period to monitor seeding progress. Upon completion of the pulsatile seeding phase, the bioreactor was transferred from the incubator to the biosafety cabinet. The valve was then removed from the bioreactor and prepared for implantation.

### Results and Conclusion

The heart valve is then implanted into the recipient patient. The results will show that the heart valve has more cells below the basement membrane of the valve which leads to better recellularization of the tissue once it is implanted.

### Example 2

This example illustrates one embodiment of the bioreactor system of the present invention.

### Methods and Materials

### Static Seeding

Silicone grip adapters were sutured proximally and distally to a decellularized, ovine pulmonary valve. A silicone plug was inserted into the bottom (proximal) grip to prevent escape of the cell suspension during static seeding. The valve was then suspended from the bioreactor cap elevator mechanism using the upper (distal) grip. Approximately 1 ml of DMEM (w/10% FBS) was then pipetted into the valve conduit via slots in the elevator mechanism to aid in closing the leaflets prior to addition of the cell suspension. Approximately 3.3×10⁶ hMSCs (human mesenchymal stem cells) were suspended in 7 ml of DMEM. The cell suspension was then pipetted into the valve conduit as described above. The bioreactor cap was then secured to the first bioreactor vessel or static seeding chamber, which was in turn filled with DMEM (˜200 ml) sufficient to cover the valve up to and including the distal suture line. The static chamber was placed in an incubator under standard cell culture conditions for 24 h.

### Vacuum Preconditioning

After 24 h static culture, the bioreactor was removed from the incubator for transferred to a second bioreactor vessel or pulsatile seeding chamber. Following removal of the silicone plug from the proximal silicone grip, an annular, silicone sheet (outer diameter equal to that of the internal diameter of the pulsatile chamber) was affixed to this grip by sandwiching it between the grip and the stainless steel magnet adapter ring. The purpose of the annular silicone sheet was to inhibit fluid flow through the external (from the perspective of the valve) holes during bellows expansion. The valve was then transferred to the pulsatile seeding chamber which was in turn filled with 500 ml DMEM (w/10% FBS). A dedicated outflow filter was affixed to the bioreactor cap. An additional outflow/inflow with external resistance was also added. This setup facilitated the generation of tunable positive pressure upon bellows compression and a negative pressure upon bellows expansion. The pulsatile chamber was then returned to the incubator and mounted to the actuator platform. The bioreactor was cyclically pulsed for 72 h using an actuator displacement rate of 0.25 cm/min for both the up and down strokes. Chamber pressure was recorded at 1 h intervals over the course of the experiment. Upon harvest, the valve was dissected. Biopsies 1-6 were sent for embedding. H&E staining was performed on sections from biopsies 2 and 5. FCl was left intact and stored in Histochoice at 4° C.

### Results and Conclusions

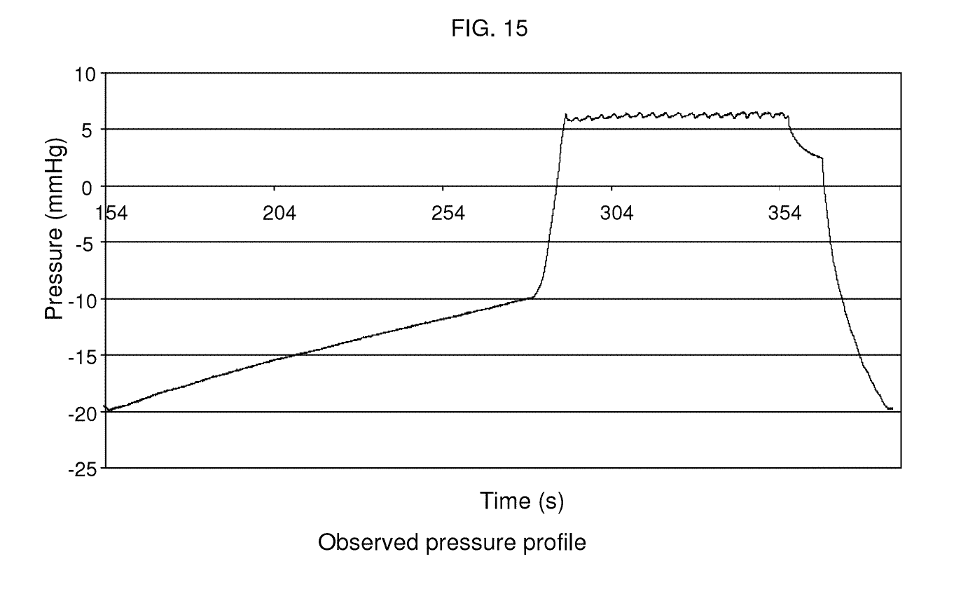

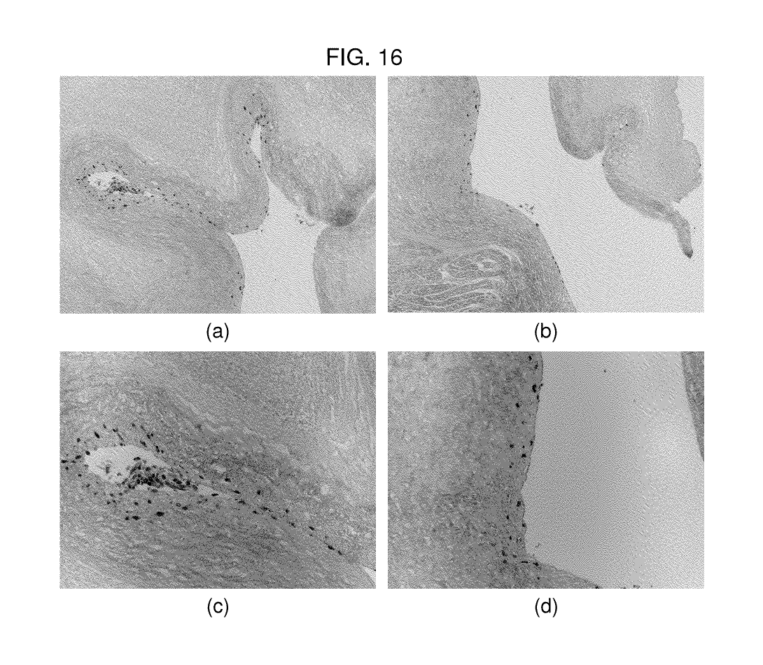

A maximum positive pressure of approximately 5 mmHg was observed during bellows compression (FIG. 15), while a maximum negative pressure of approximately −20 mmHg was observed during bellows expansion. Following 72 h vacuum preconditioning, cells had begun to infiltrate the leaflet tissue (FIG. 16). Initial static cell seeding was performed on the outflow surface of the leaflet. Cell infiltration appears to have been confined to the outer portion of the fibrosa and did not reach the spongiosa. The images in FIG. 16 show sections taken from biopsy 5. The leaflet tissue was not discernible in sections taken from biopsy 2.

### Example 3

This example illustrates tissue engineering a living heart valve using one embodiment of the disposable single use self-contained bioreactor system of the present invention.

### Materials and Methods

The bioreactor system used for heart valve tissue engineering in this example is fully disposable and intended for patient-specific, one-time use. The bioreactor of the present invention was designed to address concerns which limit practicality in translating tissue engineered constructs from the bench top to clinical practice. Specifically, this investigation demonstrated one possible filter configuration, comprising an inflow/outflow filter on the left, and a dedicated outflow filter on the right.

While the current design accommodates heart valves and other tubular structures, modifications to the attachment mechanism would permit use with other constructs. The bioreactor comprises three major assemblies, including 1) a static seeding chamber in which the initial introduction and attachment of cells is achieved, 2) a pulsatile chamber for the mechanical conditioning of seeded tissues and 3) a cap to which the construct is attached, permitting easy transfer between chambers. The static seeding chamber consists of a custom molded polystyrene cup with multiple luer ports for the addition or removal of culture medium and the addition of cell suspensions. The lower portion of the chamber is conical geometrically focus cell seeding.

The pulsatile conditioning chamber permits mechanical conditioning through the linear compression and expansion of a simple bellows. The bellows is located beneath the polystyrene valve chamber. Upon compression, culture medium is driven from the bellows into polystyrene chamber. The flow rate into the polystyrene chamber, and thus through and around the valve construct, is controlled by the rate of linear bellows compression. The chamber baseplate (the division between the polystyrene valve chamber and the bellows) comprises a large, centralized opening surrounded by numerous, concentrically arranged holes around the periphery. This hole-pattern permits central flow through the lumen of the construct, as well as flow outside the tissue. The baseplate design provides a “self-homing” mechanism to centrally position and secures valves within the chamber. This is accomplished using an annular magnet fully encased (not in contact with culture medium) within the baseplate. This works in parallel with a martensitic stainless steel ring and silicone grip sutured to the proximal end of the heart valve. As with the static chamber, the pulsatile chamber incorporates multiple luer ports, providing access for pressure monitoring and media exchange.

The polypropylene cap is designed to aid in the initial seeding of valves, transfer of valves between seeding chambers and positioning valves at the desired height within the chambers. This was accomplished through the use of an elevator system designed to accommodate a silicone grip, which is first sutured to the distal end of the heart valve or other tubular tissue construct. The silicone grip is attached to the elevator through a moderate interference fit. The position of the elevator and consequently, the vertical position of the valve, can be adjusted while the cap is affixed to either seeding chamber. Eight luer ports were incorporated into the cap. External filters, check valves and restrictors were attached to the luer ports, providing control over gas exchange within the seeding chambers. This, coupled with the rate of linear bellows compression, facilitated the use of a wide variety of cyclic pressure profiles during mechanical conditioning, including negative phase conditioning, positive phase conditioning during preliminary seeding.

Pressuer traces are shown above demonstrating pressure profiles used for (a) negative phase conditioning and (b) positive phase conditioning during seeding experiments. This shows that bioreactor system is capable of generating negative chamber pressures below −35 mmHg (c) as well as positive pressures greater than those of system circulation (d). The cycle periods were adjusted by altering the rate of bellow compression.

The efficacy of the bioreactor as a tool for the seeding of decellularized semi-lunar heart valves was been evaluated, using both commercially available human MSCs and MNCs (mononuclear cells) filtered directly from ovine bone marrow. Human MSCs (˜5.0×10⁶) were seeded directly into the lumen of decellularized ovine aortic valves (AVs). A silicone plug was used to occlude the proximal grip, preventing escape of the cell suspension during seeding and allowing cells to settle onto the outflow surface (fibrosa) of the closed leaflets. Incubation in the static seeding chamber for 24 h resulted in cell adherence to the fibrosa, though cell clusters were observed in undulations on the surface of the AVL (aortic valve leaflets). Following transfer to the pulsatile conditioning chamber, seeded valves were subjected to 72 h of negative phase conditioning (NPC) using a cyclic pressure waveform in the range +5 to −20 mmHg. Selected valves were also subjected to subsequent physiologic pressure conditioning (PPC) for an additional 10 days at +50 mmHg.

### Results and Conclusions

The application of mechanical conditioning often resulted in migration of seeded cells from the site of initial seeding (fibrosa) to the ventricularis. After 24 h static seeding, hMSCs attachment to the AVL and cell infiltration into the AVL following PPC were observed. Some clustering (large arrows) of cells was observed on the fibrosa regardless of processing. Infiltration following only NPC was observed occasionally, but not on a consistent basis. Planar biaxial testing performed in accordance with the above established methods indicated that areal strain under equibiaxial loading of NPC and PPC conditioned AVLs was nearly restored to cryopreserved state

The graphs above show the effects of mechanical conditioning on biaxial properties of the ovine AVL showing (a) aeral strain and (b) peak stretch ratio in both specimen directions.

Infiltration of MNCs into the PVL (pulmonary valve leaflets) occurred much more quickly, within 48 h of initial seeding. MNCs were filtered from 25 ml of ovine bone marrow using a newly developed bone marrow separation device (Bone Marrow MSC Separation Device, Kaneka Corporation) and seeded on decellularized ovine PVs as described above. Seeded valves were statically incubated for 24 h, followed by 24 h NPC. Substantial infiltration of MNCs occurred following this shortened protocol. Therefore, there was improved seeding response compared to commercial MSCs. Further, culture of filtered MNCs revealed a subpopulation of adherent cells exhibiting a typical MSC morphology.

A previously designed custom Real-time PCR TaqMan Array was used to track stem cell differentiation into valve interstitial cells (VICs). Gene expression patterns for BGLAP, SSP1, BMP2, BMP4, BMP7, ACAN, ENOS, PCNA, BAX, HMGB1, COL1A1, COL2A1, COL3A1, COL4A1, COL5A1, COL6A1, HSP47, VIM, ACTA2, FABP4, CD106, CD105, CD73, CD90, CD34, CD45, BCL-2, EPAS1, and GAPDH were compared for human pulmonary valve VICs, human MSCs, human articular cartilage chondrocytes and human osteocytes. CD34 gene expression was limited to VICs whereas CD106, BCL-2, and SPP1 were not expressed in VICs but detected only in MSCs, chondrocytes, and osteocytes. COL2A1 and FABP4 were exclusively expressed in NHACs. ACAN expression was only detected in osteocytes whereas BMP4 expression was absent all together. Among gene candidates actively expressed by all four groups, EPAS1 and COL4A1 served as positive VIC markers, as they were severely down regulated in osteocytes, chondrocytes, and MSCs. Fold change data demonstrated MSC phenotype maintenance with ACTA2 expression highly up regulated (43.47+/−13.43 for MSCs compared to VICs). Similarly, both COL5A1 and COL6A1 were up regulated in MSCs at 28.98+/−6.93 and 28.19+/−5.01, respectively.

The effects of decellularization on the ovine PV have extensively characterized, and histological evaluation indicated the complete removal of cell nuclei and debris following the decellularization process. Analysis of double-stranded DNA (dsDNA) concentration within the PVL demonstrated nearly complete removal (>97.5%) of nuclear material, supporting histological findings. Uniaxial tensile testing indicated significantly higher tensile strength for decellularized PVL tissue, compared to cryopreserved tissue (Table 1, p<0.04, Mann-Whitney U-test). The tensile properties of AVL tissue were unaffected by processing; however, differences in stiffness between AVL and PVL tissue were significant for all processing groups (Table 1). Differences in tensile strength between AVL and PVL tissue were also significant for fresh and decellularized tissue (Table 1).

TABLE 1
Tensile strength (UTS) and Stiffness (E) of ovine leaflet tissue under
uniaxial loading. Properties are reported in kN/m.

| | PV | | AV | |
|---|---|---|---|---|
| | UTS | E | UTS | E |
| Fresh | 1.2 (0.2) | 4.2 (1.4) | 2.3 (0.8)* | 10.8 (1.8)* |
| Cryopreserved | 1.0 (0.2) | 3.0 (1.0) | 1.9 (0.6) | 7.8 (2.8)* |
| Decellularized | 1.4 (0.3)¹ | 3.5 (0.9) | 2.0 (0.4)* | 8.9 (1.4)* |

¹Significant difference with Cryo (p < 0.04)
*Significant difference with PVL tissue (p < 0.005)

The mechanical behavior of the ovine PVL was also tested under equibiaxial loading. Reduced relaxation was observed following decellularization. Increased stretch was also observed along both specimen axes following decellularization, resulting in increased areal strain. The effects of biaxial properties of the ovine PLV showing (a) relaxation and (b) peak stretch ratio in both specimen directions.

An absence of creep was observed under all processing conditions, which is promising towards the utilization of the decellularized heart valves as valve substitute and as a scaffold for the tissue engineered heart valve (TEHV). Additional testing indicated a significant reduction in sulfated GAG concentration following decellularization, which likely contributed to the reduction in relaxation. In vitro hydrodynamic and wear testing performed on a custom built pulse duplicator indicated comparable hydrodynamic and wear performance between cryopreserved and decellularized ovine PVs. Chronic implant studies in sheep also indicate comparable hemodynamic performance between the cryopreserved and decellularized valves.

These implant studies utilizing decellularized ovine PVs also indicated sporadic in vivo re-endothelization of the leaflet and inconsistent recellularization within the basal region of the cusp. As described above, the bioreactor as provided in the present invention has demonstrated potential as a tool for accomplishing this ex vivo conditioning, with optimal processing conditions, including the ideal cell population for valve seeding and the extent of processing required yet to be explored, as demonstrated in the following example(s).

### Example 4

This example illustrates the effects of the seeding process using the bioreactor to establish a pilot cell population within the decellularized PVL using bone marrow derived cell sources

### Materials and Methods.

This example will systematically investigate the effects of the seeding process (i.e., static seeding, negative-phase conditioning) on the biology, function and mechanical behavior of decellularized heart valve constructs, leading to methods for establishing optimal pilot populations of bone marrow derived cells within the decellularized PVL. This work will also be critical in identifying potential advantages and disadvantages of specific cell sources as regards the seeding of heart valve scaffolds.
Leaflet Static Seeding: Two cell sources, both derived from bone marrow and both potentially recipient-specific, will be investigated, including 1) bone marrow filtrate, comprising MNCs filtered directly from ovine bone marrow and 2) MSCs expanded from bone marrow filtrate through additional culture. Initial experimentation will investigate the attachment behavior of MNCs and MSCs seeded under static conditions. To improve experimental control over seeding and to permit the evaluation of multiple seeding time points, individual leaflets will be excised from ovine decellularized pulmonary valves and directly seeded with either MNCs or MSCs under static conditions for time periods of 1, 3, 5, 24 and 48 h. Cell attachment will be evaluated histologically. Leaflet tissue from 10 decellularized valves will be randomly allocated such that 3 (n=3) PVLs are seeded per time point per cell source. Shorter durations (≦5 h) are clearly favorable towards the clinical processing of the TEHV as these would possibly permit overnight conditioning period and next day implantation; however, cell attachment may be sacrificed. As described above, our initial experience with these seeding techniques has demonstrated infiltration of MNCs after only 48 h (24 h static +24 h NPC). Therefore, evaluation of extended static seeding periods (i.e., 24 and 48 h) will elucidate the relative contributions of total seeding duration, static seeding duration and NPC to cell infiltration.
Whole-Valve Static Seeding: The short (≦5 h) and extended (≧24 h) time point showing the most promise in terms of initial cell attachment, and perhaps cell infiltration, will be selected for whole-valve bioreactor based seeding. MNCs or MSCs will be suspended in 10 mL of culture media and pipetted directly into the valve conduit, closing the leaflets and permitting static seeding on the outflow surface (fibrosa). A silicone plug will be used to occlude the proximal end of the valve, preventing escape of the cell suspension. Following incubation under static conditions, valves will be harvested for further evaluation. Cell attachment will be assessed through H&E staining. Terminal deoxynucleotidyl transferase dUTP nick end labeling (TUNEL) will be used to evaluate cell viability. Four combinations of cell source (MNCs, MSCs) and static seeding duration (long, short) will be evaluated. Three (n=3) decellularized PVs seeded per combination will provide sufficient tissue for histological evaluation and TUNEL.
Negative Phase Conditioning: To evaluate the contributions of NPC, additional valves will be statically seeded and subjected to subsequent NPC. During NPC, valves will be subjected to either a high (−20 mmHg) or low (−5 mmHg) negative chamber pressure to investigate the effects of pressure intensity on cell infiltration. The length of NPC will also be varied to include short (e.g. 1 h), medium (e.g., 24 h) and long (e.g., 72 h) periods. Multiple variables contribute to the complexity of this portion of the study, including cell type (MNCs vs. MSCs), static seeding duration (short vs. long), pressure intensity (low vs. high) and NPC duration (short vs. medium vs. long). To reduce the workload and amount of animal tissue required, pressure intensity will be evaluated for both cell types, but only at a single combination of static seeding duration and NPC duration (4 possible combinations). Following selection of the preferred chamber pressure, the remaining 10 combinations of cell source, static seeding duration and NPC duration will be evaluated. Cellular density (cells/μm²) and viability will be assessed through H&E staining (n=3 leaflets) and TUNEL (n=3 leaflets), respectively. Cell phenotype expression (n=3 leaflets) will be evaluated by real-time PCR and IHC. Given the heterogeneous and multi-potent nature of the seeded cells, expression of MSC, VIC, osteocyte, chondrocyte, adipocyte, hematopoietic stem cell genes will be investigated by real-time PCR. IHC will be performed using antibodies that have been verified as being effective for ovine aries for MSCs (Stro-1, CD105, CD73, CD90), VICs (αSMA, VIM, HSP47, DES, eNOS⁻, vWF⁻) and inflammatory cells (CD68, CD45, CD34). dsDNA will be quantified using our previously published methods. Bench top hydrodynamic testing (n=5 valves) will be performed using established methods to evaluate the effects of NPC on valve function. Leaflets from the same valves will subsequently be excised and subjected to biaxial mechanical testing (n=9 leaflets). In all, 9 decellularized PVs will be required per experimental group to ensure adequate tissue for the completion of the proposed study.

### Results and Conclusions

Data Analysis, Statistics, Power Calculations: Cell attachment to the surface of the leaflet will be quantified as the number of cells/μm of the fibrosa. Similarly, cell density within the leaflet will be measured as the number of cells/μm² of leaflet cross-sectional area. These measures will be further combined with TUNEL observations to quantify the attachment or infiltration of viable cells. Gene expression data determined by real-time PCR will be analyzed using the comparative C_{T} method. Hydrodynamic performance data will be collected using the pulse duplicator, including pressure drop (ΔP), effective orifice area (EOA) and regurgitant fraction. Finally, both the quasi-static and viscoelastic behavior of the leaflet will be evaluated during planar biaxial testing. Normality of collected data will be determined using Kolmogrov-Smirnoff tests, and parametric (ANOVA) or non-parametric (Kruskal-Wallis) test methods will be used depending on the distribution of data. Appropriate post-hoc analysis will be performed when indicated. Power analysis (Sigma Stat 3.5, Systat Software, Inc.) of previously collected hydrodynamic test data and biaxial test data indicated >80% statistical power using sample sizes of n=5 valves and n=9 leaflet (i.e., 3 valves), respectively. Additionally, previously collected data from quantitative morphometric measurements and dsDNA analysis indicate similar levels of statistical power at n=3 leaflets.
Expected Results, Limitations, Alternate Approaches: The studies associated with this Example will systematically investigate the contributions of static seeding and NPC towards the ex vivo generation of a pilot cell population within the decellularized pulmonary leaflet. It is anticipated that this portion of the work will result in optimized seeding protocols for this purpose. Compromises between optimal cell viability and optimal density of infused cells may be necessary. For example, high intensity seeding protocols (i.e., long duration, highly negative chamber pressures) will likely result in better cell infiltration into the PVL; however, such protocols may have negative consequences on cell viability, compared to less intense processing (i.e., short duration, low negative chamber pressures). Negative consequences on cell phenotype expression (e.g., expression of osteocyte, adipocyte or chondrocyte phenotype) are not expected, given the relatively short duration of static and NPC portions of the seeding process. However, if undesired differentiation is observed, steps will be taken to delay differentiation of the initial cell population, or drive differentiation towards the desired phenotype (i.e., VICs), through modifications to the culture media. It is anticipated that a heterogeneous population of MNCs would be filtered directly from bone marrow and the phenotypic expression is consistent with MSCs in populations of expanded cells. The established methods through previous examples will be used to evaluate valve hydrodynamic and planar behavior of seeded valves. Negative consequences associated with the seeding process are not anticipated; however, maintenance of in vitro valve function is critical to in vivo performance, warranting investigation. The observation of any detrimental effects would be instructive towards the selection of appropriate seeding strategies for subsequent work.

Custom primers and probes (Applied Biosystems, Foster City, Calif.) will be required for real-time PCR analysis given the current commercial availability of ovine markers. Many Ovis aries gene target sequences are sourced from previously published data available through NCBI GenBank and RefSeq databases. Targets not available within NCBI are derived in a predictive manner using RefSeq and GenBank sequence data for species closely related to Ovis aries (i.e. Bos Taurus and Equus caballus) for local alignment analysis in BLASTn. Given high degrees of cross-species sequence homogeneity, corresponding Ovis aries expressed sequence tags (ESTs) can be effectively linked in order to establish target specificity on the basis of a high percent query coverage, low E score, and maximum sequence identity to known cross-species targets with high certainty as defined within the output of NCBIs BLASTn search algorithm. All sequence information is used for custom ovine primer/probe development according to specific PCR reaction criteria listed within Primer Express software (Applied Biosystems, Foster City, Calif.).

The preferred method of MNC separation from ovine bone marrow is direct filtration. While the filtration system is marketed for MSC separation, a fraction of the hematopoietic cells present in the marrow are also collected, though red blood cells are effectively removed. Given the relative proportions of MSCs and hematopoietic cells in bone marrow, MSCs constitute only a fraction the collected cells. The composition of the MNC cell population obtained through direct filtration of marrow will be compared with that of other separation techniques (e.g. density gradients) prior to undertaking the proposed studies. While filtration is attractive for the clinical setting due to simplicity, MNC recovery through the use of Percoll or Ficoll gradients would constitute only a minor deviation from anticipated clinical protocols. These methods would be acceptable for use in these studies if they offered advantages in terms of cell recovery and viability over the filtration system. Preliminary experiments with bone marrow filtration and seeding have been performed, in which filtration of bone marrow aliquots generally occurred within 30 min of harvest. It may be necessary to increase the heparinization of aspirated bone marrow at the time of harvest should coagulation become detrimental to the filtration process during the overnight shipping of the bone marrow to another facility to complete the study associated with this example. Alternative methods of MNC separation from bone marrow, as described above, will be explored if direct filtration remains impractical. As a final failsafe, local sources of ovine bone marrow will be pursued if challenges persist (e.g., two vet schools are approximately a 2 h drive from CMH).

### Example 5

This example illustrates a study to determine the maximal extent of ex vivo maturation of a seeded cell population and the resulting potential for restoration of leaflet composition and tissue remodeling.

### Materials and Methods

Completion of this Example will elucidate the role of extended bioreactor based conditioning on the ex vivo maturation of a pilot cell population within the decellularized PVL. The study described is designed to investigate the potential for 1) continued matrix repopulation through proliferation of seeded MNCs or MSCs, 2) differentiation of seeded cells into VICs and 3) associated downstream effects on ECM restoration and remodeling. Decellularized pulmonary valves will be seeded with MNCs or MSCs pursuant to optimized protocols determined through the completion of Example 1 and subjected to further bioreactor conditioning under physiologic pressures simulating systolic loading in the pulmonary circulation (i.e., 35-40 mmHg) for periods of 2, 4 and 6 weeks. Cellular density (n=3 leaflets), viability (n=3 leaflets) and phenotype expression (n=3 leaflets) will be evaluated as described above, as will dsDNA concentration (n=3 leaflets) and valve mechanical behavior (n=5 valves). Assays for sulfated GAGs (n=3 leaflets, Blyscan Assay), collagen (n=3 leaflets, Sircol Assay) and elastin (n=3 leaflets, Fastin Assay) will be performed and compared with previously collected data for the decellularized ovine pulmonary valve to quantify the extent of ECM restoration and remodeling. To correlate valve composition with valve mechanical behavior, these assays will be performed on tissues previously subjected to hydrodynamic and planar biaxial testing. Histological staining will also be used to further evaluate restoration of GAGs (e.g., Movat's Pentachrome, Alcian Blue) and deposition of new collagen (e.g., Masson's Trichrome) within the PVL. In all, 9 decellularized PVs will be required per experimental group to ensure adequate tissue for the completion of the proposed studies.

### Results and Conclusions

Data Analysis, Statistics, Power Calculations: Evaluations associated with Example 5 are similar to those in Example 4, with the addition of assays for sulfated GAG, collagen and elastin concentration. These ECM components will be quantified in μg/mg dry tissue. Similar statistical methods will be used to analyze these data as those described above. Previous analysis of sulfated GAG content within the PVL indicated >80% statistical power using sample sizes of n=3 leaflets (Sigma Stat 3.5, Systat Software, Inc.).
Expected Results, Limitations, Alternate Approaches: Increased repopulation of the decellularized PVL and differentiation of seeded cells into VICs leading to the restoration of GAG content and ECM remodeling is expected for valves seeded with expanded MSCs. Should this not occur, alterations will be made to the mechanical conditioning environment (e.g., chamber pressure, fluid flow rate, cyclic compression rate). Media supplementation with appropriate growth and differentiation factors may also prove beneficial. Considering the paracrine signaling mechanism presumed to drive in vivo recellularization of MNC seeded constructs, the continued repopulation of MNC tissues over periods of extended ex vivo conditioning is less certain, as there will be no circulating cells to recruit following initial seeding; however, even a negative outcome will be beneficial in selecting optimized seeding protocols and cell source for the TEHV. The effects of extended conditioning under physiologic pressure on valve function and mechanical integrity will largely depend on the extent of ECM restoration that is achieved. Normal function is expected in the presence of compositional restoration and ECM remodeling; however, failure to achieve this would likely result in valvular insufficiency and deteriorated structural integrity of the leaflet. Either result will provide useful knowledge towards the development of clinically applicable seeding protocols for the TEHV, as shortened processing times offer inherent regulatory advantages and are of greater practicality in the clinical setting. In the event that extended conditioning periods are accompanied by significant negative consequences to valve biology or function, or do not offer appreciable benefits, the in vivo study proposed in Example 3 will be modified to avoid unnecessary expense and animal sacrifice.

### Example 6

This example quantitatively assesses the effects of a seeded cell population on the in vivo recellularization, ECM restoration and performance of the TEHV.

This experimental series will be undertaken to address questions regarding the necessity of a fully recellularized valve prior to implantation. Clearly, valve substitutes must be fully functional at the time of implantation; however, for valve scaffolds that have been proven functional (i.e., decellularized heart valves), it remains unknown whether the strategy of full ex vivo recellularization with a fully differentiated cell population offers significant advantages over the more time effective approach of simply generating a “pilot” cell population prior to implantation. Decellularized ovine PVs will be seeded with recipient-specific, bone marrow derived cells using the optimized protocols developed in Examples 1 and 2 to establish either a “pilot” or a mature cell population within the leaflet. Seeded valves will be implanted in the right ventricular outflow tract (RVOT) of juvenile sheep for 6 months. Contingent upon the results from previous studies, up to 4 experimental groups will be evaluated to account for relevant combinations of cell type (i.e., filtered MNCs, expanded MSCs) and pre-implant recellularization level (i.e., pilot, mature). Prior to explant, transesophageal echocardiography (TEE) and cardiac catheterization/angiography will be performed to evaluate in vivo valve performance, therefore hydrodynamic testing will not be performed on explanted valves. Recellularization, cell phenotype expression, mechanical behavior and compositional restoration of the leaflet will be evaluated as described above. Eight (8) PVs will be implanted per group to ensure adequate tissue availability for the proposed analyses.
Expected Results, Limitations, Alternate Approaches: We do not expect significant advantages in terms of terminal repopulation, compositional restoration or valve function for valves seeded to a mature state prior to implantation, compared to those implanted with only a pilot population. Rather, we anticipate full recellularization and compositional restoration of seeded valves regardless of the initial level of processing. While not directly addressed in this project, subsequent studies will investigate the in vivo mechanisms (e.g. paracrine signaling, proliferation/differentiation of seeded cells) through which this recellularization occurs, which are likely different for MNC and MSC seeded constructs. Implants will be performed by an experienced surgical team using well developed techniques and a robust ovine strain. Excellent survivability is expected; however, the implantation of 8 valves per group will provide sufficient tissue to mitigate the impact of unexpected early death on our ability complete the proposed evaluations. The ovine implant model represents the current gold-standard for the pre-regulatory evaluation of cardiovascular devices, including valve substitutes. This is largely due to a propensity towards calcification of implanted of implanted devices, thus the ovine model will offer the most rigorous evaluation of the TEHV. A major attribute to our processing paradigm is the use of recipient-specific cells to initially seed allogeneic, decellularized heart valve scaffolds. Thus, utilizing a small animal model to initially evaluate the in vivo response to seeded tissues would not be worthwhile or cost effective, as this would require that the entire processing paradigm be altered to account for the change in species.

1. A bioreactor system comprising:
a. one or more bioreactor vessels 1; wherein said bioreactor vessel 1 comprises, a proximal end, 15 a distal end 5, a continuous cylindrical side wall 10, one or more ports 11 on the continuous cylindrical side wall 10,;
b. a bioreactor vessel cap 40, wherein said cap 40 comprises a housing for an elevator mechanism 55, an elevator mechanism 60 and an attachment element 56; wherein said cap 40 is removably attachable to the proximal end 15 of the bioreactor vessel(s 1); and
c. one or more tissue retainers 35 secured to said elevator mechanism 60 and housing 55.

2. The bioreactor system of claim 1, comprising two bioreactor vessels 1, 20.

3. The bioreactor system of claim 2, wherein a first bioreactor vessel 1 has a portion on the distal end 5 that narrows in diameter 6 from the circumference of said continuous cylindrical side wall 10 at an angle to a flat plane 7 that has a narrower circumference than that of said continuous side wall 10.

4. The bioreactor system of claim 3, wherein the circumference of said flat plane 7 is 1% to 80% of the circumference of said continuous cylindrical side wall 10.

4. The bioreactor system of claim 3, wherein said first bioreactor vessel 1 provides for a concentration of cells to congregate at said flat plane 7 for static cell seeding of a tissue.

5. The bioreactor system of claim 3, wherein a second bioreactor vessel 20 has a plurality of openings 26 on the distal end 25 of said second bioreactor vessel 20 to allow fluid to flow in and out of said second bioreactor vessel 20

6. The bioreactor system of claim 5, wherein the distal end 25 of the second bioreactor vessel 20 further comprises a homing device 75 that centers the said tissue retainer 35 at the distal end 25 of the second bioreactor vessel 20 through an attraction means.

7. The bioreactor system of claim 6, wherein said homing device 75 is a magnet.

8. The bioreactor system of claim 5, wherein said distal end 25 of said second bioreactor vessel 20 is connected to a bellows system 65 to provide a pulsatile pressure waveform.

9. A bioreactor vessel 1 comprising a continuous cylindrical side wall 10 and two ends, 5, 15, where one end narrows on an angle to a closed flat plane 7, wherein the diameter of the closed flat plane is less than the diameter of said continuous side wall 10.

10. The bioreactor vessel of claim 9, wherein the diameter of said closed flat plane 7 is from about 1% to 80% smaller than the diameter of said continuous side wall 10.

11. The bioreactor vessel of claim 10, wherein said narrowing allows for a concentration of cells to aggregate at the bottom of said bioreactor vessel 1.

12. A bioreactor cap 40 comprising a surface area that is large enough to cover the proximal opening 16, 23 of a bioreactor vessel 1, 20, two opposing faces 45, 46, and a plurality of openings 50 on the top face 45.

13. The bioreactor cap 40 of claim 12, further comprising a stepped portion 42 with a smaller diameter than said surface area of the cap extending upwards from the top face 45 of the cap 40.

14. The bioreactor cap 40 of claim 12, wherein the plurality of openings 50 allow for the use of external resistors or filters to alter the outflow and pressure.

15. The bioreactor cap 40 of claim 12, further comprising an elevator mechanism 60 with a housing 55 for the upward and downward motion of a tissue 36 within a bioreactor vessel 1, 20.

16. An elevator mechanism system 51, comprising a housing 60, an elevator mechanism 55, having attachment means and a control knob 63.

17. The elevator mechanism system 51 of claim 16, wherein said elevator mechanism 55 has a threaded element 62.

18. The elevator mechanism system 51 of claim 16, wherein said attachment means 56 are for attaching the elevator mechanism system 51 to a tissue retainer 35.

19. The elevator mechanism system 51 of claim 17, wherein said control knob 63 allows for the upward or downward movement of a tissue 36.

20. The elevator mechanism system 51 of claim 16, wherein said housing 60 attaches to a bioreactor cap 40.

21. The elevator mechanism system 51 of claim 18, further comprising a tissue 36 attached to said tissue retainer 35.

22. A method of cell seeding and tissue conditioning, comprising:
a. placing a tissue 36 between two spaced tissue retainers 35
b. connecting one of the tissue retainers 35 to an elevator mechanism system 51 comprising a housing 55 and an elevator mechanism 60 attached to a bioreactor cap 40;
c. attaching the cap 40 to a first bioreactor vessel 1 having a distal end 5 that narrows to a flat plane 7 having a smaller diameter than that of the first bioreactor vessel 1;
d. providing cells for seeding to said distal end 5 of said first bioreactor 1 such that said cells contact said tissue 36;
e. removing the bioreactor cap 40 from said first bioreactor vessel 1 and attaching said cap 40 to a second bioreactor vessel 20 having a distal end 25 with a plurality of openings 26;
f. attaching a bellows 65 to the distal end 25 of said second bioreactor vessel 20 containing fluid; and
g. allowing the fluid from the bellows 65 to move in and out of the second bioreactor vessel 20 contacting said tissue 36.

23. The method of claim 22, wherein said tissue 36 is decellularized.

24. The method of claim 22, further comprising a homing device 75 in one of said tissue retainers 35 and a complementary receiving device on the distal end 25 of said second bioreactor vessel 20.

25. The method of claim 22, wherein the elevator mechanism system 51 allows the tissue 36 to be centered within the second bioreactor vessel 20.

26. The method of claim 22, wherein the tunable pressure within the second bioreactor vessel 20 is from about −5 mmHg to about 200 mmHg.

27. The method of claim 24, wherein said homing device 75 facilitates the media flow through both the inside and outside of said tissue 36.
